SEQUENCE LISTING (33 sequences)

SEQ ID NO: 1 - DNA, 50 bp, Homo sapiens
  source 1..50: mol_type = genomic DNA
gttgcaggtcactgtagcgggacttcttttggttttctttctctttgggg

SEQ ID NO: 2 - DNA, 50 bp, Homo sapiens
  source 1..50: mol_type = genomic DNA
attcctgaagctgacagcattcgggccgagatgtctcgctccgtggcctt

SEQ ID NO: 3 - DNA, 50 bp, Homo sapiens
  source 1..50: mol_type = genomic DNA
agtgacagcggcccgctggagaggaagcccgagagctgccgcgcgcctgc

SEQ ID NO: 4 - DNA, 50 bp, Homo sapiens
  source 1..50: mol_type = genomic DNA
gctgaagtgaaaacgagaccaaggtctagctctactgttggtacttatga

SEQ ID NO: 5 - DNA, 50 bp, Homo sapiens
  source 1..50: mol_type = genomic DNA
ctcttcttcttaaacaaaccacaaacggatgtgagggaaggaaggtgttt

SEQ ID NO: 6 - DNA, 50 bp, Homo sapiens
  source 1..50: mol_type = genomic DNA
actgcccgcgggagccgccgccgccgccgccgcgcccgccatggacgtcc

SEQ ID NO: 7 - DNA, 50 bp, Homo sapiens
  source 1..50: mol_type = genomic DNA
agagcgagctgcggccgtggcagctgcacggctcctggccccggagcatg

SEQ ID NO: 8 - DNA, 50 bp, Homo sapiens
  source 1..50: mol_type = genomic DNA
agttaagctggtggcagcagccgaggccaccaagaggcaacgggcggcag

SEQ ID NO: 9 - DNA, 50 bp, Homo sapiens
  source 1..50: mol_type = genomic DNA
aaagcgagcgacatgtccctttggggagcagtccctctgcaccccagagt

SEQ ID NO: 10 - DNA, 50 bp, Homo sapiens; identical to SEQ ID NO: 2
  source 1..50: mol_type = genomic DNA

SEQ ID NO: 11 - DNA, 50 bp, Homo sapiens
  source 1..50: mol_type = genomic DNA
actcgcgcgcacggagcgacgacacccccgcgcgtgcacccgctcgggac

SEQ ID NO: 12 - DNA, 22 bp, synthetic construct
  source 1..22: mol_type = other DNA
gtctcctctgacttcaacagcg

SEQ ID NO: 13 - DNA, 22 bp, synthetic construct
  source 1..22: mol_type = other DNA
accaccctgttgctgtagccaa

SEQ ID NO: 14 - DNA, 23 bp, synthetic construct
  source 1..23: mol_type = other DNA
ccttgaaagctcagaactcggag

SEQ ID NO: 15 - DNA, 22 bp, synthetic construct
  source 1..22: mol_type = other DNA
tgctgcgttagcatgagttggc

SEQ ID NO: 16 - DNA, 22 bp, synthetic construct
  source 1..22: mol_type = other DNA
cagcttagccaggtggaaatcc

SEQ ID NO: 17 - DNA, 23 bp, synthetic construct
  source 1..23: mol_type = other DNA
gtcgttggagatgacaagttccg

SEQ ID NO: 18 - DNA, 21 bp, synthetic construct
  source 1..21: mol_type = other DNA
gtccaacaatgtgacccagat

SEQ ID NO: 19 - DNA, 20 bp, synthetic construct
  source 1..20: mol_type = other DNA
acctcaacgatatgcagccg

SEQ ID NO: 20 - DNA, 23 bp, synthetic construct
  source 1..23: mol_type = other DNA
tagagacaaactgtgagtggtca

SEQ ID NO: 21 - DNA, 23 bp, synthetic construct
  source 1..23: mol_type = other DNA
gggcatgttagtctgtgtttctg

SEQ ID NO: 22 - DNA, 22 bp, synthetic construct
  source 1..22: mol_type = other DNA
cgctacctttggcgaagtctct

SEQ ID NO: 23 - DNA, 22 bp, synthetic construct
  source 1..22: mol_type = other DNA
ctggctctgtatgctgcgagtt

SEQ ID NO: 24 - DNA, 22 bp, synthetic construct
  source 1..22: mol_type = other DNA
ctcaggaagaggagtcggaagt

SEQ ID NO: 25 - DNA, 22 bp, synthetic construct
  source 1..22: mol_type = other DNA
acacaggcttctgcggctcatt

SEQ ID NO: 26 - DNA, 22 bp, synthetic construct
  source 1..22: mol_type = other DNA
ttcgcccttagcgtgaagatgg

SEQ ID NO: 27 - DNA, 22 bp, synthetic construct
  source 1..22: mol_type = other DNA
tagtgctccagcagctcgaaga

SEQ ID NO: 28 - DNA, 22 bp, synthetic construct
  source 1..22: mol_type = other DNA
tgtgaccgagtcctctggaagt

SEQ ID NO: 29 - DNA, 23 bp, synthetic construct
  source 1..23: mol_type = other DNA
gcctcaaatgtggcaaagacagg

SEQ ID NO: 30 - DNA, 19 bp, synthetic construct
  source 1..19: mol_type = other DNA
tattgccgcccagaagagc

SEQ ID NO: 31 - DNA, 21 bp, synthetic construct
  source 1..21: mol_type = other DNA
gcttgatctccttgcgtagag

SEQ ID NO: 32 - DNA, 24 bp, synthetic construct
  source 1..24: mol_type = other DNA
ccactgaaaaagatgagtatgcct

SEQ ID NO: 33 - DNA, 23 bp, synthetic construct
  source 1..23: mol_type = other DNA
ccaatccaaatgcggcatcttca

(19) United States
(12) Patent Application Publication
(10) Pub. No.: US 2026/0297628 A1
(43) Pub. Date: Oct. 1, 2026

(54) CHIMERIC POLYPEPTIDE SYSTEMS AND METHODS OF GENE REGULATION

(71) Applicant: FUNDAÇÃO D. ANNA DE SOMMER CHAMPALIMAUD E DR. CARLOS MONTEZ CHAMPALIMAUD - CENTRO DE INVESTIGAÇÃO DA, Lisboa (PT)

(72) Inventors: Zhifen YANG, Menlo Park, CA (US); Maggie L. BOBBIN, San Mateo, CA (US); Vitaly BALAN, Foster City, CA (US); Rona HARARI-STEINFELD, Sunnyvale, CA (US); Francesco M. MARINCOLA, Half Moon Bay, CA (US)

(21) Appl. No.: 18/880,537

(22) PCT Filed: Jun. 29, 2023
(86) PCT No.: PCT/IB2023/056783

§ 371 Date: Dec. 31, 2024

Related U.S. Application Data
(60) Provisional application No. 63/357,728, filed on Jul. 1, 2022.

(51) Int. Cl. C12N 15/90; A61K 35/17; C12N 5/0783; C12N 9/22; C12N 15/11

(52) U.S. Cl. CPC C12N 15/907; A61K 35/17; C12N 5/0636; C12N 9/226; C12N 15/11; C12N 2310/20; C12N 2510/00

(57) ABSTRACT

Certain aspects of the present disclosure provides systems, compositions, and methods for regulating expression or activity of a target protein in a cell. In some cases, the present disclosure provides a system comprising an actuator moiety capable of complexing with a target polynucleotide sequence, to regulate expression or activity of the target protein. The actuator moiety can be heterologous to the cell. The actuator moiety can be activatable upon exposing the cell to an external stimulus. Upon the exposure of the cell to the external stimulus, the actuator moiety can be activated to regulate expression or activity of the target protein.

This application claims the benefit of U.S. Patent Application No. 63/357,728, filed on Jul. 1, 2022, which is incorporated herein by reference in its entirety.

### BACKGROUND ART

Cancers (e.g., neoplasm, tumor) are a large family of diseases that involve abnormal cell growth in a number of bodily tissues. Cancers can invade or spread to other parts of the body. As leading causes of death worldwide, cancers accounting for about 10 million deaths annually. Non-limiting examples of bodily tissues invaded by cancers include lung, prostate, colorectal, stomach, liver, breast, colon, rectum, cervix, and thyroid. With a goal of treating or controlling cancers, different therapies have been developed, e.g., small molecules, antibodies, and adoptive cell therapies (e.g., cellular immunotherapy).

### SUMMARY OF INVENTION

The present disclosure provides methods and systems for adoptive cell therapies to treat a subject having or is suspected of having a condition, such as cancer. Methods and systems of the present disclosure may be used to, e.g., enhance activity (e.g., anti-tumor activity) of cellular immunotherapy (e.g., cancer therapies using autologous or allogeneic immune cells).

In an aspect, the present disclosure provides a system for regulating expression or activity of a target protein of a cell, the system comprising an actuator moiety capable of complexing with a target polynucleotide sequence in the cell, wherein the actuator moiety is heterologous to the cell, and wherein the target polynucleotide sequence is endogenous to the cell, wherein the complexing effects a change in the expression or activity of the target protein by at least about 10% as compared to that in a control cell, wherein the complexing is sufficient to effect the change without editing the target polynucleotide sequence, and wherein the target protein comprises one or more members selected from the group consisting of thymocyte selection-associated high mobility group box protein (TOX), suppressor of cytokine signaling (SOCS), basic leucine zipper transcription factor ATF-like (BATF), inhibitor of DNA binding/differentiation (ID), T-box transcription factor (TBX), c-Jun.

In another aspect, the present disclosure provides a system for regulating expression or activity of a target protein of a cell, the system comprising an actuator moiety capable of complexing with a target polynucleotide sequence in the cell, wherein the actuator moiety is heterologous to the cell and is activatable for the complexing upon exposure of the cell to an external stimulus, and wherein the target polynucleotide sequence is endogenous to the cell, wherein, upon the exposure, the actuator moiety is activated for the complexing to effect a change in the expression or activity of the target protein, wherein the complexing is sufficient to effect the change without editing the target polynucleotide sequence, and wherein the target protein comprises Src homology 2 domain containing inositol phosphatase (SHIP) or beta-2-microglobulin (B2M), and TGF beta receptor (TGFbR).

In another aspect, the present disclosure provides a system comprising a guide nucleic acid molecule designed to bind a target polynucleotide sequence for regulating expression or activity of a target protein of a cell, wherein the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein, wherein the target protein is selected from the group consisting of thymocyte selection-associated high mobility group box protein (TOX), suppressor of cytokine signaling (SOCS), Src homology 2 domain containing inositol phosphatase (SHIP), basic leucine zipper transcription factor ATF-like (BATF), beta-2-microglobulin (B2M), inhibitor of DNA binding/differentiation (ID), T-box transcription factor (TBX), c-Jun, and TGF beta receptor (TGFbR).

In another aspect, the present disclosure provides a system comprising an actuator moiety capable of binding a target polynucleotide sequence for regulating expression or activity of a target protein of a cell, wherein the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein, wherein the target protein is selected from the group consisting of thymocyte selection-associated high mobility group box protein (TOX), suppressor of cytokine signaling (SOCS), Src homology 2 domain containing inositol phosphatase (SHIP), basic leucine zipper transcription factor ATF-like (BATF), beta-2-microglobulin (B2M), inhibitor of DNA binding/differentiation (ID), T-box transcription factor (TBX), c-Jun, and TGF beta receptor (TGFbR).

In another aspect, the present disclosure provides a method for regulating expression or activity of a target protein of a cell, comprising: (a) forming a complex comprising an actuator moiety and a target polynucleotide sequence in the cell, wherein the actuator moiety is heterologous to the cell, and wherein the target polynucleotide sequence is endogenous to the cell; and (b) in response to the forming, inducing a change in the expression or activity of the target protein by at least about 10% as compared to that in a control cell, wherein the forming of the complex is sufficient to effect the change without editing the target polynucleotide sequence, wherein the target protein comprises one or more members selected from the group consisting of thymocyte selection-associated high mobility group box protein (TOX), suppressor of cytokine signaling (SOCS), basic leucine zipper transcription factor ATF-like (BATF), inhibitor of DNA binding/differentiation (ID), T-box transcription factor (TBX), c-Jun, and TGF beta receptor (TGFbR).

In another aspect, the present disclosure provides a method for regulating expression or activity of a target protein of a cell, comprising: (a) exposing the cell to an external stimulus to activate an actuator moiety to complex with a target polynucleotide sequence in the cell, wherein the actuator moiety is heterologous to the cell, and wherein the target polynucleotide sequence is endogenous to the cell; and (b) in response to the complexing, inducing a change in the expression or activity of the target protein, wherein the forming of the complex is sufficient to effect the change without editing the target polynucleotide sequence, wherein the target protein comprises Src homology 2 domain containing inositol phosphatase (SHIP) or beta-2-microglobulin (B2M).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

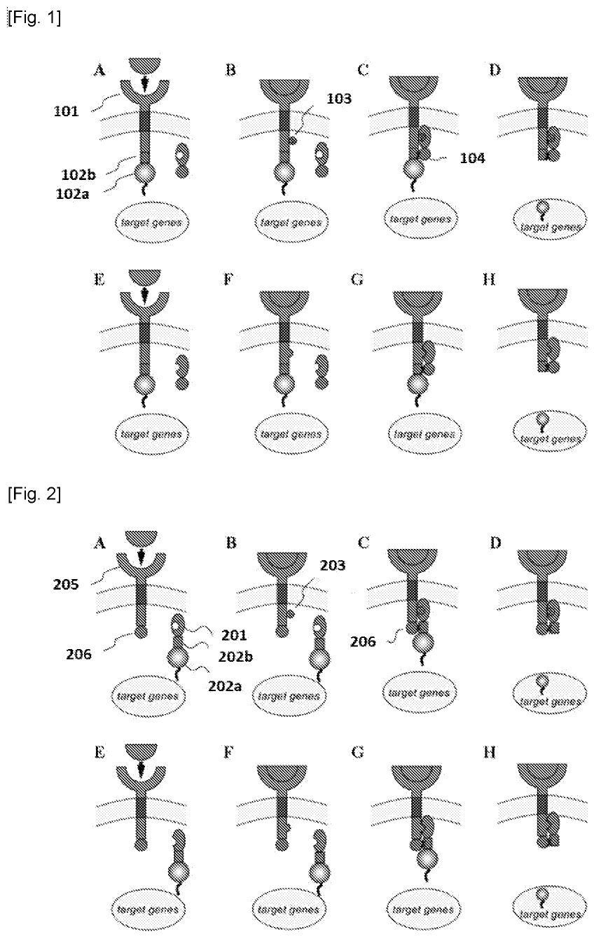

### FIG. 1

FIGS. 1A-1D illustrate schematically the release of an actuator moiety from a GMP in a system comprising a receptor which undergoes phosphorylation; FIGS. 1E-1H illustrate schematically the release of an actuator moiety from a GMP in a system comprising a receptor which undergoes a conformational change.

### FIG. 2

FIGS. 2A-2D illustrate schematically the release of an actuator moiety from a GMP in a different system comprising a receptor which undergoes phosphorylation upon ligand binding; FIGS. 2E-2H illustrate schematically the release of an actuator moiety from a GMP in a system comprising a receptor which undergoes a conformational change.

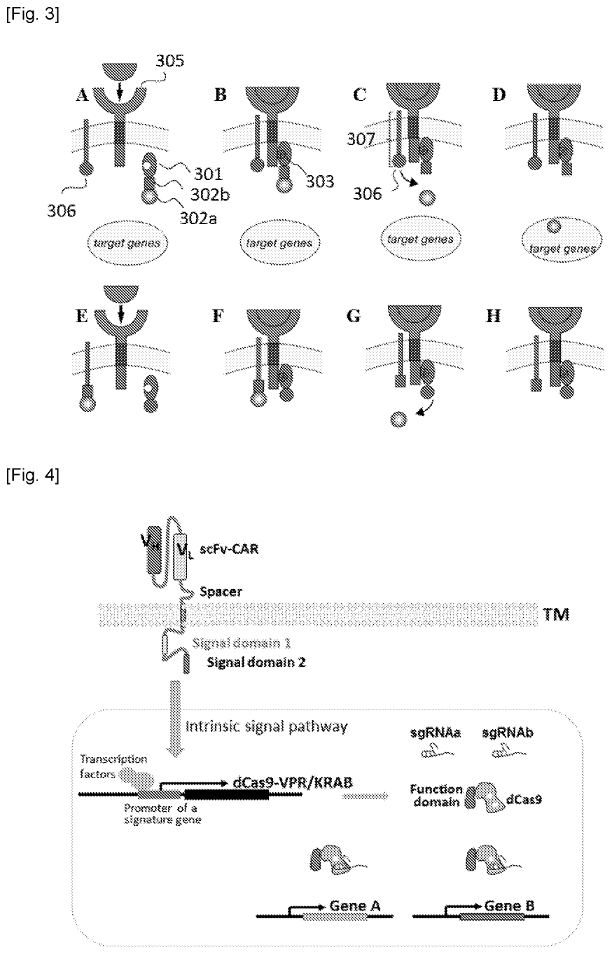

### FIG. 3

FIGS. 3A-3D illustrate schematically the release of an actuator moiety from a GMP in a system comprising at least two adaptor polypeptides and a receptor which undergoes phosphorylation; FIGS. 3E-3H illustrate schematically the release of an actuator moiety from a GMP in a system comprising at least two adaptor polypeptides and a receptor which undergoes a conformational change.

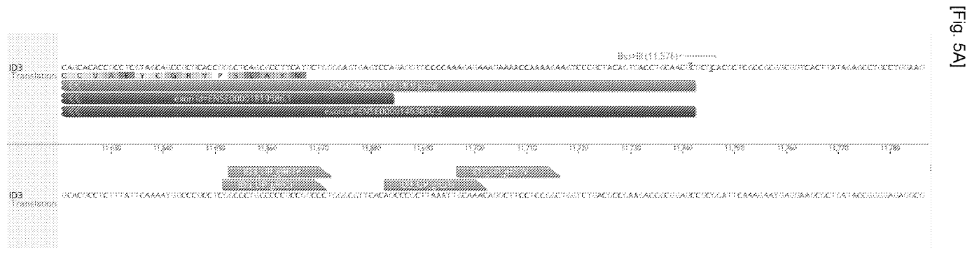

### FIG. 4

FIG. 4 illustrates schematically conditionally inducible expression of an actuator moiety via a chimeric receptor signaling.

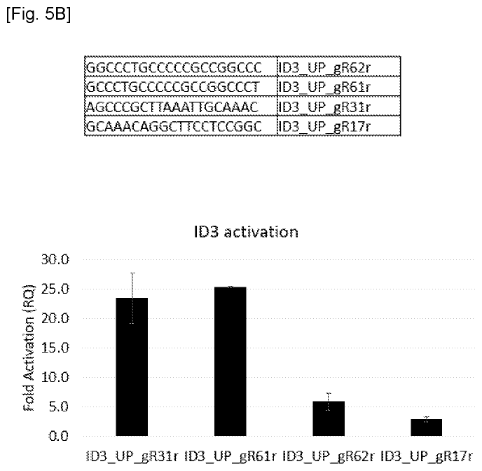

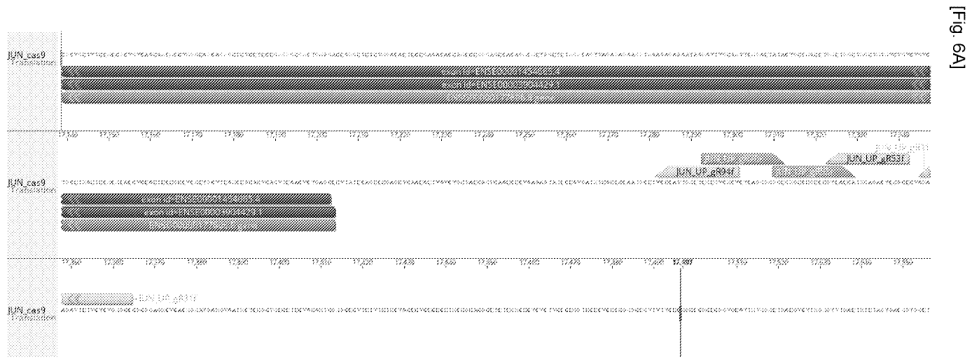

### FIG. 5

FIGS. 5A and 5B show examples of guide nucleic acid molecules against ID3 gene and relative expression levels of ID3 upon regulation by the system and method of the present disclosure.

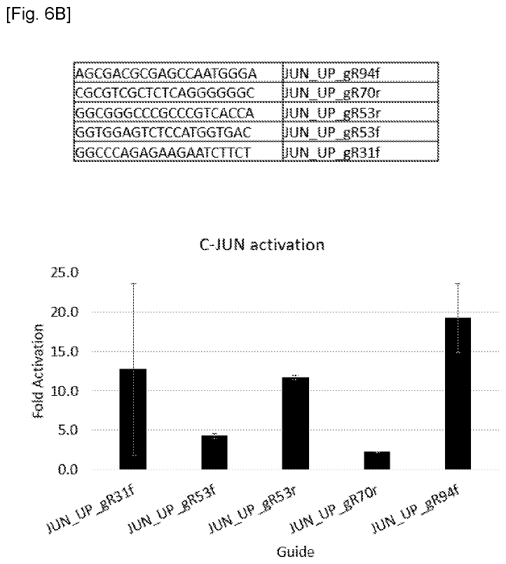

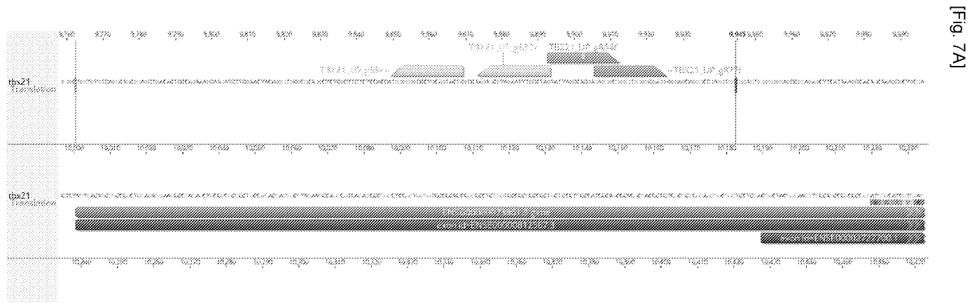

### FIG. 6

FIGS. 6A and 6B show examples of guide nucleic acid molecules against c-Jun gene and relative expression levels of c-Jun upon regulation by the system and method of the present disclosure.

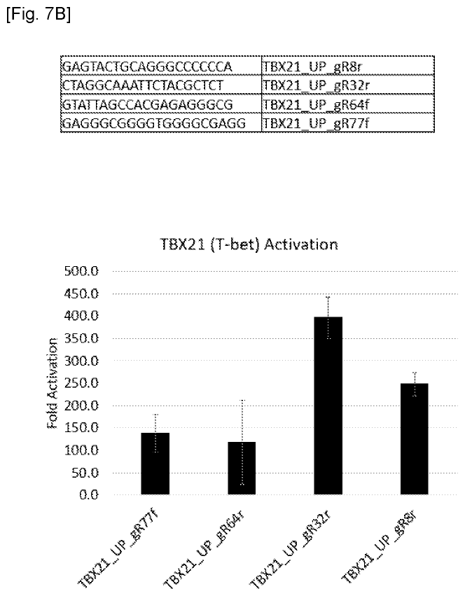

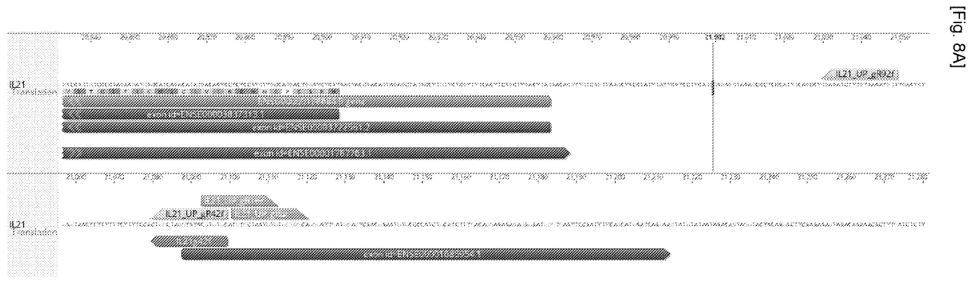

### FIG. 7

FIGS. 7A and 7B show examples of guide nucleic acid molecules against TBX21 gene and relative expression levels of TBX21 upon regulation by the system and method of the present disclosure.

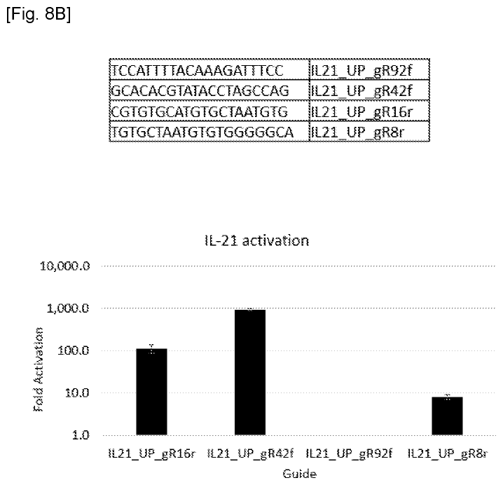

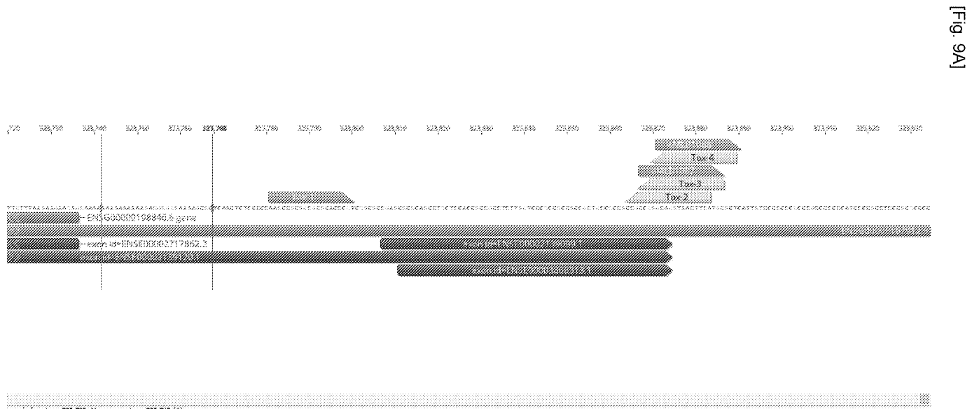

### FIG. 8

FIGS. 8A and 8B show examples of guide nucleic acid molecules against IL-21 gene and relative expression levels of IL-21 upon regulation by the system and method of the present disclosure.

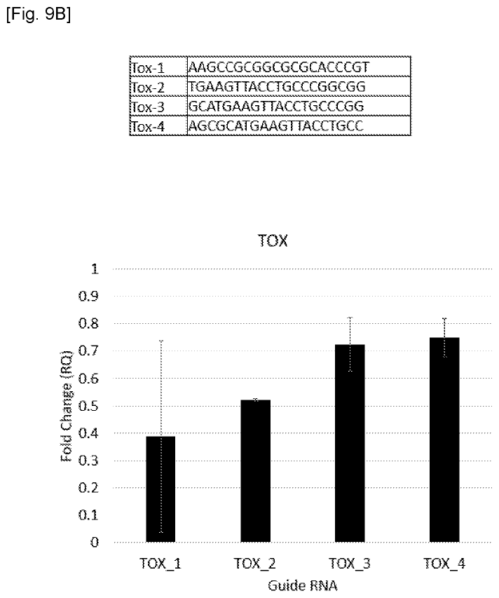

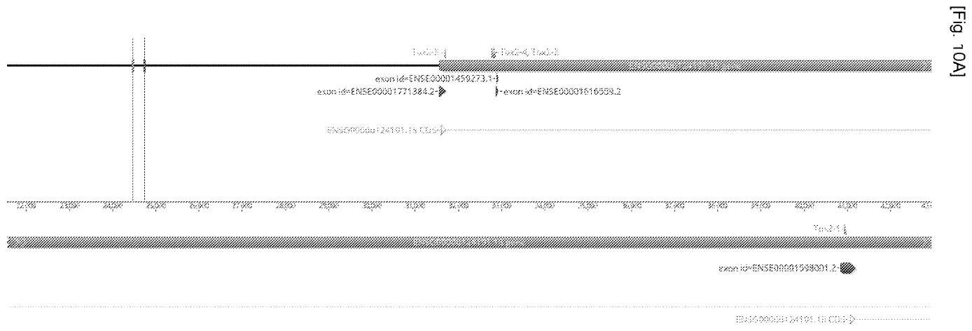

### FIG. 9

FIGS. 9A and 9B show examples of guide nucleic acid molecules against TOX1 gene and relative expression levels of TOX1 upon regulation by the system and method of the present disclosure.

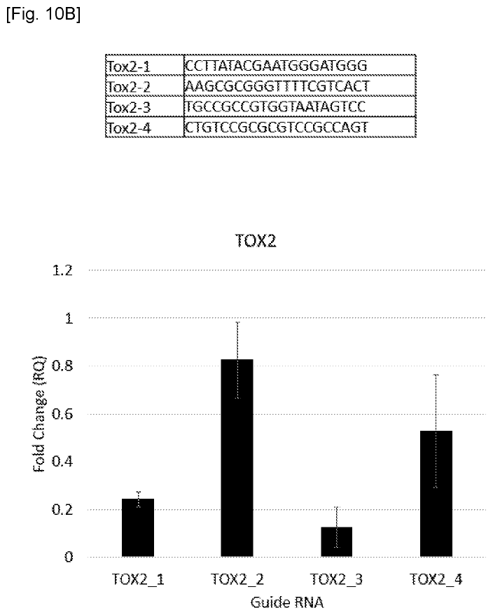

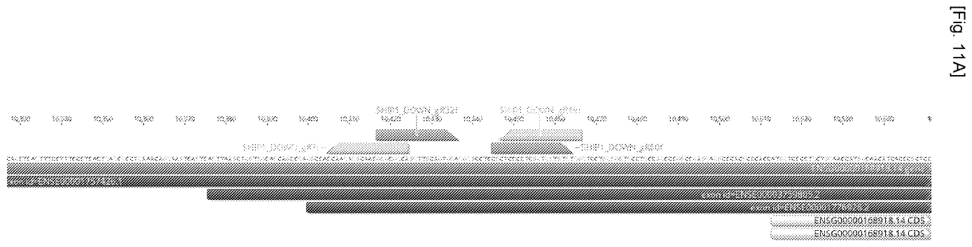

### FIG. 10

FIGS. 10A and 10B show examples of guide nucleic acid molecules against TOX2 gene and relative expression levels of TOX2 upon regulation by the system and method of the present disclosure.

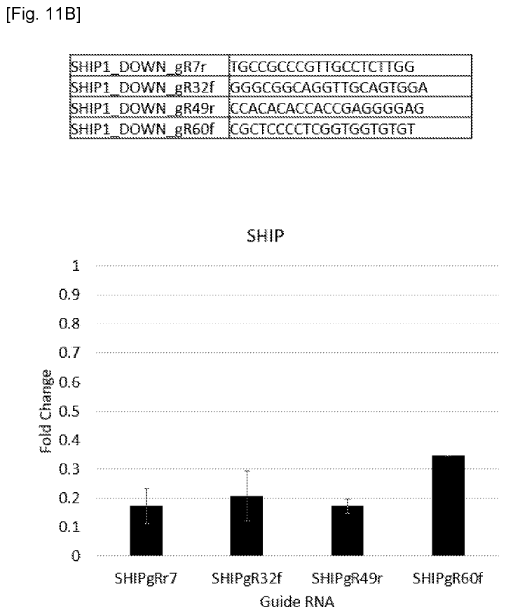

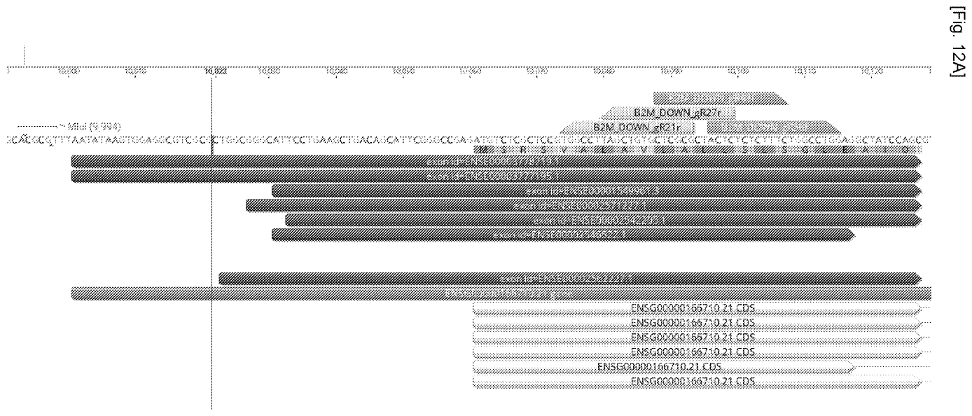

### FIG. 11

FIGS. 11A and 11B show examples of guide nucleic acid molecules against SHIP1 gene and relative expression levels of SHIP1 upon regulation by the system and method of the present disclosure.

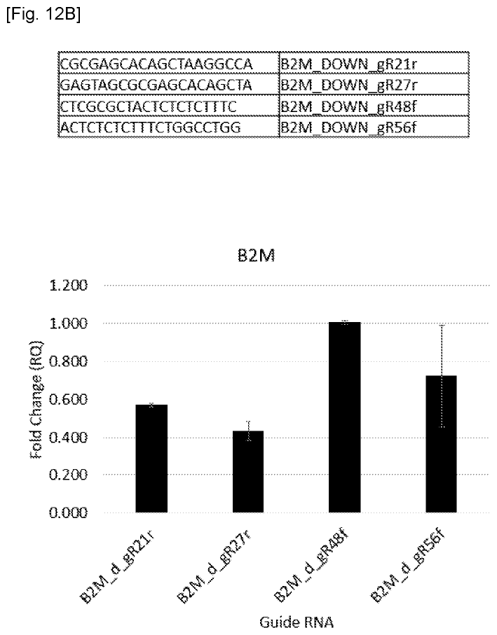

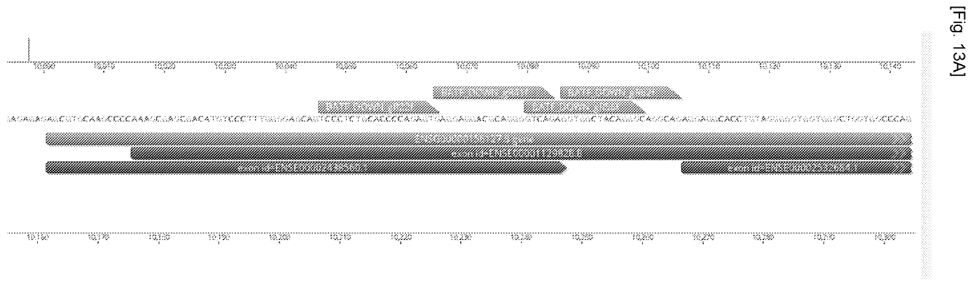

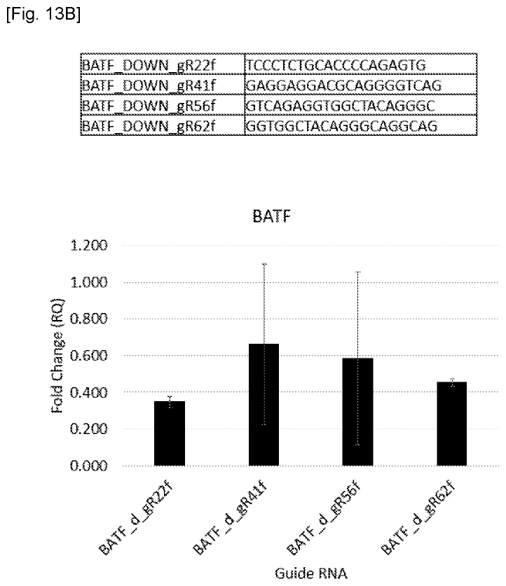

### FIG. 12

FIGS. 12A and 12B show examples of guide nucleic acid molecules against B2M gene and relative expression levels of B2M upon regulation by the system and method of the present disclosure.

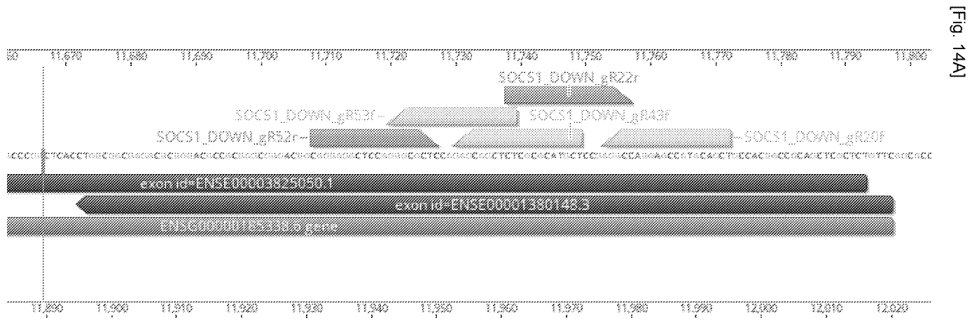

### FIG. 13

FIGS. 13A and 13B show examples of guide nucleic acid molecules against BATF gene and relative expression levels of BATF upon regulation by the system and method of the present disclosure.

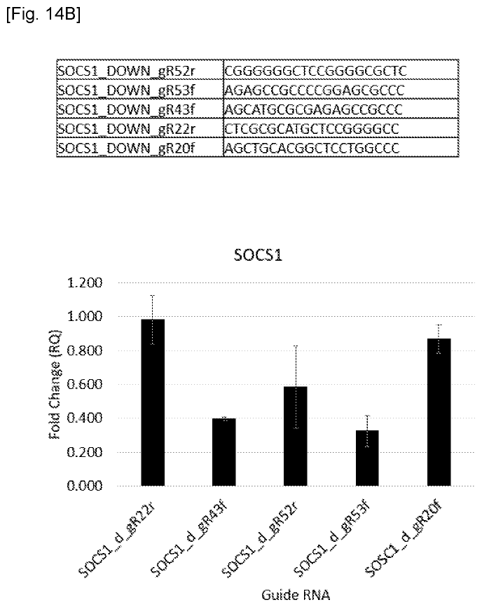

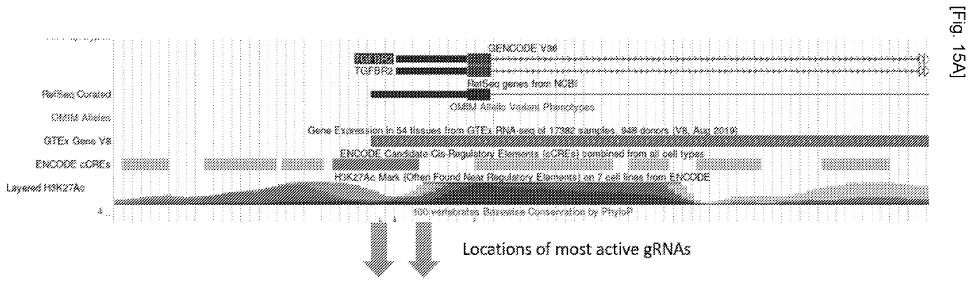

### FIG. 14

FIGS. 14A and 14B show examples of guide nucleic acid molecules against SOCS1 gene and relative expression levels of SOCS1 upon regulation by the system and method of the present disclosure.

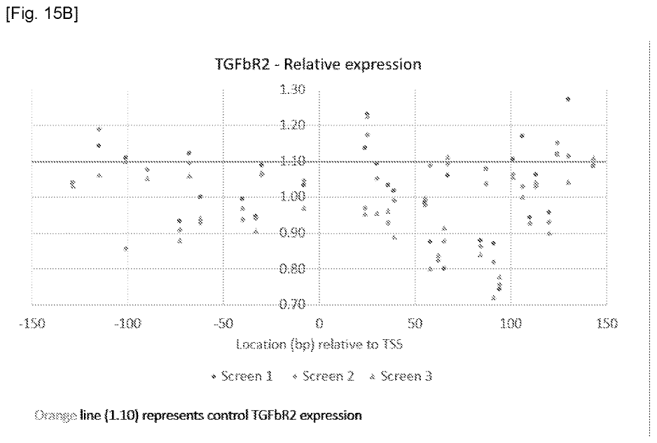

### FIG. 15

FIG. 15A illustrates example target polynucleotide sequences of a target gene encoding TGFbR2 that can be targeted by one or more guide RNAs, and FIG. 15B illustrates modified expression levels of the target gene by the system and method of the present disclosure.

### DESCRIPTION OF EMBODIMENTS

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a,” “an,” and “the” include plural references unless the context clearly dictates otherwise.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The term “hematopoietic stem cells,”, “hematopoietic progenitor cells,” or “hematopoietic precursor cells,” as used interchangeably herein, generally refers to cells which are committed to a hematopoietic lineage but are capable of further hematopoietic differentiation (e.g., into T cells) and include, multipotent hematopoietic stem cells (hematoblasts), myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells).

The term “immune cell” or “lymphocyte” generally refers to a differentiated hematopoietic cell. Non-limiting examples of an immune cell can include a T cell, an NK cell, a monocyte, an innate lymphocyte, a tumor-infiltrating lymphocyte, a macrophage, a granulocyte, etc.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G] dCTP, [TAMRA] dCTP, [JOE] ddATP, [R6G] ddATP, [FAM] ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethyl-rhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or flurescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The terms “transfection” or “transfected” refer to introduction of a nucleic acid into a cell by non-viral or viral-based methods. The nucleic acid molecules may be gene sequences encoding complete proteins or functional portions thereof.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “downregulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “expression cassette,” “expression construct,” or “expression vector” refers to a nucleic acid that includes a nucleotide sequence such as a coding sequence and a template sequence, and sequences necessary for expression of the coding sequence. The expression cassette can be viral or non-viral. For instance, an expression cassette includes a nucleic acid construct, which when introduced into a host cell, results in transcription and/or translation of a RNA or polypeptide, respectively. Antisense constructs or sense constructs that are not or cannot be translated are expressly included by this definition. One of skill will recognize that the inserted polynucleotide sequence need not be identical, but may be only substantially similar to a sequence of the gene from which it was derived.

A “plasmid,” as used herein, generally refers to a non-viral expression vector, e.g., a nucleic acid molecule that encodes for genes and/or regulatory elements necessary for the expression of genes. A “viral vector,” as used herein, generally refers to a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The term “promoter,” as used herein, refers to a polynucleotide sequence capable of driving transcription of a coding sequence in a cell. Thus, promoters used in the polynucleotide constructs of the disclosure include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions, developmental conditions, or drug or chemical conditions.

The terms “complement,” “complements,” “complementary,” and

“complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity.

Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods.

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “gene modulating polypeptide” or “GMP,” as used herein, refers to a polypeptide comprising at least an actuator moiety capable of regulating expression or activity of a gene and/or editing a nucleic acid sequence. A GMP can comprise additional peptide sequences which are not involved in modulating gene expression, for example cleavage recognition sites, linker sequences, targeting sequences, etc.

The terms “actuator moiety,” “actuator domain,” and “gene modulating domain,” as used herein, refers to a moiety which can regulate expression or activity of a gene and/or edit a nucleic acid sequence, whether exogenous or endogenous. An actuator moiety can regulate expression of a gene at the transcription level and/or the translation level. An actuator moiety can regulate gene expression at the transcription level, for example, by regulating the production of mRNA from DNA, such as chromosomal DNA or cDNA. In some embodiments, an actuator moiety recruits at least one transcription factor that binds to a specific DNA sequence, thereby controlling the rate of transcription of genetic information from DNA to mRNA. An actuator moiety can itself bind to DNA and regulate transcription by physical obstruction, for example preventing proteins such as RNA polymerase and other associated proteins from assembling on a DNA template. An actuator moiety can regulate expression of a gene at the translation level, for example, by regulating the production of protein from mRNA template. In some embodiments, an actuator moiety regulates gene expression by affecting the stability of an mRNA transcript. In some embodiments, an actuator moiety regulates expression of a gene by editing a nucleic acid sequence (e.g., a region of a genome). In some embodiments, an actuator moiety regulates expression of a gene by editing an mRNA template. Editing a nucleic acid sequence can, in some cases, alter the underlying template for gene expression.

The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a receptor polypeptide or an adaptor polypeptide) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

As used herein, “fusion” can refer to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as alexa fluor dyes, Cyanine3 dye, Cyanine5 dye.

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion in a Cas protein. An fusion protein can be a non-native sequence in a Cas protein.

As used herein, “non-native” can refer to a nucleic acid or polypeptide sequence that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “antibody” generally refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e. IgA, IgG, IgM, IgD and IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)2, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The term “antigen binding moiety” or “antigen binding domain,” as used interchangeably herein, generally refers to a construct exhibiting preferential binding to a specific target antigen. An antigen binding domain can be a polypeptide construct, such as an antibody, modification thereof, fragment thereof, or a combination thereof. The antigen binding domain can be any antibody as disclosed herein, or a functional variant thereof. Non-limiting examples of an antigen binding domain can include a murine antibody, a human antibody, a humanized antibody, a camel Ig, a shark heavy-chain-only antibody (VNAR), Ig NAR, a chimeric antibody, a recombinant antibody, or antibody fragment thereof. Non-limiting examples of antibody fragment include Fab, Fab′, F(ab)′2, F (ab)′3, Fv, single chain antigen binding fragment (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody.

The term “enhanced activity,” “increased activity,” or “upregulated activity” generally refers to activity of a moiety of interest (e.g., a polynucleotide or a polypeptide) that is modified to a level that is above a normal level of activity of the moiety of interest in a host strain (e.g., a host cell). The normal level of activity can be substantially zero (or null) or higher than zero. The moiety of interest can comprise a polypeptide construct of the host strain. The moiety of interest can comprise a heterologous polypeptide construct that is introduced to or into the host strain. For example, a heterologous gene encoding a polypeptide of interest can be knocked-in (KI) to a genome of the host strain for enhanced activity of the polypeptide of interest in the host strain.

The term “reduced activity,” “decreased activity,” or “downregulated activity” generally refers to activity of a moiety of interest (e.g., a polynucleotide or a polypeptide) that is modified to a level that is below a normal level of activity of the moiety of interest in a host strain (e.g., a host cell). The normal level of activity is higher than zero. The moiety of interest can comprise an endogenous gene or polypeptide construct of the host strain. In some cases, the moiety of interest can be knocked-out or knocked-down in the host strain. In some examples, reduced activity of the moiety of interest can include a complete inhibition of such activity in the host strain.

The term “subject,” “individual,” or “patient,” as used interchangeably herein, generally refers to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The term “treatment” or “treating” generally refers to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

As used herein, “administer,” “administering,” “administration,” and derivatives thereof refer to the methods that may be used to enable delivery of agents or compositions to the desired site of biological action. These methods include, but are not limited to parenteral administration (e.g., intravenous, subcutaneous, intraperitoneal, intramuscular, intravascular, intrathecal, intranasal, intravitreal, infusion and local injection), transmucosal injection, oral administration, administration as a suppository, and topical administration. Administration is by any route, including parenteral. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transplantation, etc.

The term “effective amount” or “therapeutically effective amount” generally refers to the quantity of a composition, for example a composition (e.g., one or more unit doses) as disclosed herein, that is sufficient to result in a desired activity upon administration to a subject in need thereof. Within the context of the present disclosure, the term “therapeutically effective” generally refers to that quantity of a composition that is sufficient to delay the manifestation, arrest the progression, relieve or alleviate at least one symptom of a disorder treated by the methods of the present disclosure.

### I. Introduction

Immune cells (e.g., T cells, NK cells) can be engineered to exhibit a specific affinity to one or more specific antigens (e.g., cancer or tumor antigens) for adoptive immunotherapy for treatment of cancers (e.g., solid tumors, lymphoma, etc.). In some cases, the immune cells can be engineered to express heterologous receptors (e.g., chimeric antigen receptors or “CAR”, engineered T cell receptor (TCR), etc.) capable of binding to one or more specific antigens, thereby targeting cancer cells in a subject.

However, therapeutic efficacy of the engineered immune cells can be limited by, for example, poor trafficking, limited persistence in serum of the subject, rapid exhaustion, or inhibitory activity of the subject's cancer cells or immune cells against the engineered immune cells. In some cases, a number of recombinant cytokines (e.g., interleukin or “IL”) can be administered to the subject along with the engineered immune cells to improve their efficacy (e.g., cytotoxicity activity, persistence, proliferation). However, co-administration of such recombinant cytokines can also exhibit unwanted adverse effects, e.g., oligemia, nausea, hepatic dysfunction, systemic toxicity, and even death. In some cases, immune cell stemness (e.g., characterized by the capacity to self-renew, the multipotency, and/or the persistence of proliferative potential) can be reduced during the engineering of T cells to express heterologous receptors as disclosed herein), thereby limiting the therapeutic efficacy of the engineered immune cells (e.g., in vivo).

Thus, there remains a significant unmet need for using heterologous cytokines to enhance efficacy of adoptive immunotherapy for treating, e.g., cancers, but with suppressed or reduced degree of the adverse effects.

### II. Systems for Gene Regulation

In one aspect, the present disclosure provides a system for regulating expression or activity of a target protein of a cell. The system can comprise an actuator moiety capable of complexing with a target polynucleotide sequence in the cell. The actuator moiety can be heterologous to the cell. In some examples, at least a portion of the amino acid sequence of the actuator moiety can be heterologous to the cell. At least a portion (e.g., all of) the target polynucleotide sequence can be endogenous to the cell. The complexing (e.g., formation of a complex comprising at least the actuator moiety and the target polynucleotide sequence) can effect a change in the expression or activity of the target protein (e.g., by at least 10%) as compared to an expression or activity of the target protein in a control cell (or a comparable cell). In some cases, the complexing can be sufficient to effect the change in the expression or activity of the target protein without editing (e.g., gene editing, such as insertion, deletion, substitution, mutation, etc.) at least a portion of the target polynucleotide sequence.

In some cases, the change (e.g., increase, decrease) in the expression or activity level of the target protein (e.g., endogenous protein) can occur (or can be observed) in vitro, ex vivo, or in vivo.

The target polynucleotide sequence can be operatively coupled to a gene encoding the target protein. The target polynucleotide sequence can be part of a coding region (e.g., exon) of the gene encoding the target protein. The target polynucleotide sequence can be part of a non-coding region (e.g., intron, promoter, transcription start site (TSS), etc.) of the gene encoding the target protein.

The target protein can comprise at least about 1, 2, 3, 4, 5, 6, 7, 8, 9, 10, or more proteins (e.g., different types of proteins). The target protein can comprise at most about 10, 9, 8, 7, 6, 5, 4, 3, 2, or 1 protein(s) (e.g., different types of proteins). Non-limiting examples of the target protein can include thymocyte selection-associated high mobility group box protein (TOX, e.g., TOX1, TOX2, TOX3, TOX4), suppressor of cytokine signaling (SOCS, e.g., SOCS1, SOCS2, SOCS3, SOCS4, SOCS5, SOCS6, SOCS7, CISH), Src homology 2 domain containing inositol phosphatase (SHIP, e.g., SHIP1, SHIP2, SHIP3), basic leucine zipper transcription factor ATF-like (BATF), beta-2-microglobulin (B2M), inhibitor of DNA binding/differentiation (ID, e.g., ID1, ID2, ID3, ID4), c-Jun, T-box transcription factor (TBX, e.g., TBX1, TBX2, TBX3, TBX4, TBX5, TBX6, TBX7, TBX8, TBX9, TBX10, TBX11, TBX12, TBX13, TBX14, TBX15, TBX16, TBX17, TBX18, TBX19, TBX20, TBX21 or T-Bet, TBX22, TBR1), interleukin (IL, such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36), and transforming growth factor beta receptor (TGFbR). Non-limiting examples of TGFbR include Type I TGFbR (e.g., ALK1, ALK2, ALK3, ALK4, ALK5, ALK6, ALK7), Type II TGFbR (e.g., TGFbR2, BMPR2, ACVR2A, ACVR2B, AMHR2), and Type III TGFbR (e.g., TGFbR3).

In some cases, the target protein may not be a secretory protein. In some cases, the target protein may not be a cytokine. In some examples, the target protein may not be an IL protein. Alternatively, the target protein may comprise an IL protein.

In some embodiments, the target protein can be operatively coupled to stemness (e.g., involved in activating, maintaining, or prolonging stemness) of a cell (e.g., an immune cell, such as a T cell). For example, one or more proteins provided in Table 1 can be regulated (e.g., activated) to enhance or prolong stemness of engineered immune cells, as disclosed herein, to thereby enhance or prolong therapeutic efficacy of the engineered immune cells.

TABLE 1
Target proteins involved in stemness of a cell (e.g., an immune cell).

| Gene target | Abbreviation | Implication | Mechanism of action |
|---|---|---|---|
| inhibitor of DNA- | ID3 | Stemness | Master regulator of T cell |
| binding 3 | | | memory differentiation induced |
| | | | by TGF-b. |
| c-Jun | c-Jun | Prevention of | Heterodimer component |
| | | exhaustion | together with c-Fos of the AP-1 |
| | | | complex that prevents ex- |
| | | | haustion upon tonic stimulation |
| | | | and terminal differentiation |
| | | | driving expression of IL-2 |
| T-box transcription | TBX21/T-bet | Stemness | Maintenance of cytokine |
| factor-21 | | | production and proliferation and |
| | | | progenitor function |
| Interleukin 21 | IL-21 | Survival/ | Modulates effector function of |
| | | expansion | CD8+ T cells |

In some cases, the change in the expression or activity of the target protein as provided herein can promote one or more features comprising (i) maintenance of stemness of a cell (e.g., an immune cell, such as a T cell), (ii) enhancing survival of the cell, and/or (iii) enhancing expansion of the cell.

In some cases, the change in the expression or activity of the target protein can activate stemness of a cell as disclosed herein (e.g., an immune cell, such as a T cell). In some cases, the change in the expression or activity of the target protein can maintain stemness of a cell, such that the stemness is prolonged (e.g., in terms of time) by at least or up to about 5%, at least or up to about 10%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 200%, at least or up to about 300%, at least or up to about 400%, at least or up to about 500%, at least or up to about 600%, at least or up to about 700%, at least or up to about 800%, at least or up to about 900%, at least or up to about 1,000%, at least or up to about 2,000%, at least or up to about 3,000%, at least or up to about 4,000%, or at least or up to about 5,000%, as compared to a control cell without the system as disclosed herein.

In some cases, the stemness of the cell as disclosed herein can be observed upon at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, at least or up to about 4 weeks, at least or up to about 1 month, at least or up to about 2 months, at least or up to about 3 months, at least or up to about 4 months, at least or up to about 5 months, or at least or up to about 6 months after inducing the complexing as disclosed herein (e.g., that after activating the actuator moiety, or that after binding of the ligand to the ligand binding domain of the chimeric receptor).

In some cases, the stemness of the cells as disclosed herein can be ascertained (or can be measured) in vitro, ex vivo, or in vivo.

In some cases, the change in the expression or activity of the target protein can enhance survival (e.g., in terms of time that the cell remains alive) of the cell as disclosed herein (e.g., an immune cell, such as a T cell) by at least or up to about 5%, at least or up to about 10%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 200%, at least or up to about 300%, at least or up to about 400%, at least or up to about 500%, at least or up to about 600%, at least or up to about 700%, at least or up to about 800%, at least or up to about 900%, at least or up to about 1,000%, at least or up to about 2,000%, at least or up to about 3,000%, at least or up to about 4,000%, or at least or up to about 5,000%, as compared to a control cell without the system as disclosed herein.

In some cases, the survival of the cell as disclosed herein (e.g., an immune cell, such as a T cell) can be observed upon at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, at least or up to about 4 weeks, at least or up to about 1 month, at least or up to about 2 months, at least or up to about 3 months, at least or up to about 4 months, at least or up to about 5 months, or at least or up to about 6 months after inducing the complexing as disclosed herein (e.g., that after activating the actuator moiety, or that after binding of the ligand to the ligand binding domain of the chimeric receptor).

In some cases, the survival of the cells as disclosed herein can be ascertained (or can be measured) in vitro, ex vivo, or in vivo.

In some cases, the change in the expression or activity of the target protein can enhance expansion (e.g., in terms of proliferation) the cell as disclosed herein (e.g., an immune cell, such as a T cell) by at least or up to about 5%, at least or up to about 10%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 200%, at least or up to about 300%, at least or up to about 400%, at least or up to about 500%, at least or up to about 600%, at least or up to about 700%, at least or up to about 800%, at least or up to about 900%, at least or up to about 1,000%, at least or up to about 2,000%, at least or up to about 3,000%, at least or up to about 4,000%, or at least or up to about 5,000%, as compared to a control cell without the system as disclosed herein.

In some cases, the expansion of the cell as disclosed herein (e.g., an immune cell, such as a T cell) can be observed upon at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, at least or up to about 4 weeks, at least or up to about 1 month, at least or up to about 2 months, at least or up to about 3 months, at least or up to about 4 months, at least or up to about 5 months, or at least or up to about 6 months after inducing the complexing as disclosed herein (e.g., that after activating the actuator moiety, or that after binding of the ligand to the ligand binding domain of the chimeric receptor).

In some cases, the expansion of the cell as disclosed herein can be ascertained (or can be measured) in vitro, ex vivo, or in vivo.

In some cases, a population of cells (e.g., a population of immune cells, such as a population of T cells) treated by the systems and methods disclosed herein can yield a proportion (e.g., percentage) of memory T cells (e.g., as indicated by being CD45RO positive) that is higher than that in a comparable population of cells that has not been treated by the systems and methods, by at least or up to about 1%, at least or up to about 2%, at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%. For example, a population of CAR-T cells treated by the systems and methods disclosed herein can yield 50% memory T cells, while a comparable population of CAR-T cells that has not been treated by the systems and methods can yield 10% memory T cells, thus the former being higher than the latter by 40%.

In some cases, in a population of cells (e.g., a population of immune cells, such as a population of T cells) treated by the systems and methods disclosed herein, central memory T cells (TCMs) in the population can be more abundant than effector memory T cells (TEMs) by at least or up to about 1%, at least or up to about 2%, at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%. TCMs can be CD45RO positive and CD62L positive. TEMs can be CD45RO positive and CD62L negative.

In some cases, a population of cells (e.g., a population of immune cells, such as a population of T cells) treated by the systems and methods disclosed herein can yield a proportion (e.g., percentage) of central memory T cells (TCMs) that is higher than that in a comparable population of stem cells that has not been treated by the systems and methods, by at least or up to about 1%, at least or up to about 2%, at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%. For example, a population of CAR-T cells treated by the systems and methods disclosed herein can yield 25% TCMs, while a comparable population of stem cells that has not been treated by the systems and methods can yield 15% TCMs, thus the former being higher than the latter by 10%.

In some cases, a population of cells (e.g., a population of immune cells, such as a population of T cells) treated by the systems and methods disclosed herein can yield a proportion (e.g., percentage) of stem memory T cells (TSCM, e.g., as indicated by being CD45RO negative and CD62L positive) that is at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%.

In some cases, a population of cells (e.g., a population of immune cells, such as a population of T cells) treated by the systems and methods disclosed herein can yield a proportion (e.g., percentage) of stem memory T cells (TSCMs, e.g., as indicated by being CD45RO negative and CD62L positive) that is higher than that in a comparable population of stem cells that has not been treated by the systems and methods, by at least or up to about 1%, at least or up to about 2%, at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%. For example, a population of CAR-T cells treated by the systems and methods disclosed herein can yield 50% TSCMs, while a comparable population of stem cells that has not been treated by the systems and methods can yield 10% TSCMs, thus the former being higher than the latter by 40%.

In some cases, a population of cells (e.g., a population of immune cells, such as a population of T cells) treated by the systems and methods disclosed herein can yield a proportion (e.g., percentage) of stem memory T cells (TSCM, e.g., as indicated by being CD45RO negative and CD62L positive) that is at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%.

In some cases, the type(s) of T cells (e.g., memory T cells, TCM, TEM, TSCM) or a proportion thereof as disclosed herein can be observed upon at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, at least or up to about 4 weeks, at least or up to about 1 month, at least or up to about 2 months, at least or up to about 3 months, at least or up to about 4 months, at least or up to about 5 months, or at least or up to about 6 months after inducing the complexing as disclosed herein (e.g., that after activating the actuator moiety, or that after binding of the ligand to the ligand binding domain of the chimeric receptor).

In some cases, the types of T cells or a proportion thereof as disclosed herein can be ascertained (or can be measured) in vitro, ex vivo, or in vivo.

In some embodiments, the target protein can be operatively coupled to a function of the engineered cell (e.g., CAR-T cells) as disclosed herein. For example, one or more proteins provided in Table 2 can be regulated (e.g., repressed) to improve one or more functions of engineered immune cells with a chimeric receptor (e.g., CAR, engineered TCR).

TABLE 2
Target proteins involved in one or more functions of engineered immune cells.

| GENE TARGET | Abbreviation | Implication | Mechanism of action |
|---|---|---|---|
| Thymocyte | TOX1 | Exhaustion | Induced by NFAT in |
| Selection Associated | | | exhausted cells |
| High Mobility | | | |
| Group Box | | | |
| TOX High Mobility | TOX2 | Exhaustion | Induced by NFAT in |
| Group Box Family | | | exhausted cells |
| Member 2 | | | |
| Suppressor of | SOCS-1 | Potency | Negative regulator of |
| cytokine signaling 1 | | | cytokine signaling |
| Src homology 2 | SHIP-1 | Exhaustion | suppressed by miR-155. |
| (SH2) domain | | (Terminal | In mice the expression is |
| containing inositol | | differentiation) | SHIP-1 suppressed Phf19 |
| polyphosphate | | | which promote terminal |
| 5-phosphatase 1 | | | differentiation of T cells |
| basic leucine zipper | BATF | Exhaustion | TCR-induced AP-1 |
| factor | | | transcription factor that induces |
| | | | expression of immune |
| | | | regulatory genes such as |
| | | | PD-1 reducing proliferation |
| | | | and cytokine production. |
| | | | Expression can be as- |
| | | | sociated with CD39^{high}, |
| | | | TIM-3^{high} TIL. |
| Beta-2 | B2M | Immunogenicity | Component of MHC class I |
| Microglobulin | | | molecules |
| TGF beta receptor 2 | TGFbR2 | Potency | TGFbR2 reduces differ- |
| | | (Terminal | TGFb ligand binding to |
| | | differentiation) | entiation of T cells to |
| | | | cytotoxic Th1 cells |

In some cases, the change in the expression or activity of the target protein as provided herein can promote one or more features comprising (i) reduced exhaustion of the engineered immune cell, (ii) enhanced cytokine production by the engineered immune cell, (iii) enhanced cytotoxicity of the engineered immune cell against a population of target cells, and/or (iv) enhanced differentiation of immune cells into immune cell subtypes (e.g., from naïve T cells to T helper cells).

In some cases, the change in the expression or activity of the target protein as provided herein can reduce exhaustion of the engineered immune cell. Exhaustion of immune cells (e.g., T cells) can be ascertained by detecting a presence of one or more exhaustion markers, such as, for example, CTLA-4, BTLA, PD-1, GITR, VISTA, TIGIT, LAG-3, and TIM-3. In some examples, a population of engineered immune cells (e.g., T cells) treated by the systems and methods disclosed herein can exhibit a proportion (e.g., percentage) of exhausted immune cells (e.g., exhausted T cells) that is lower than that in a comparable population of immune cells that has not been treated by the systems and methods, by at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%.

In some cases, the immune cell exhaustion markers as disclosed herein can be measured upon at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, at least or up to about 4 weeks, at least or up to about 1 month, at least or up to about 2 months, at least or up to about 3 months, at least or up to about 4 months, at least or up to about 5 months, or at least or up to about 6 months after inducing the complexing as disclosed herein (e.g., that after activating the actuator moiety, or that after binding of the ligand to the ligand binding domain of the chimeric receptor).

In some cases, the immune cell exhaustion markers as disclosed herein can be ascertained (or can be measured) in vitro, ex vivo, or in vivo.

In some cases, the change in the expression or activity of the target protein as provided herein can enhance cytokine production (e.g., expression) by the engineered immune cell. Such cytokine may not and need not be the same protein as the target protein whose expression or activity is directly regulated by the systems and methods as disclosed herein. Such cytokine can be an endogenous cytokine of the cell. Such cytokine whose production is enhanced may not be the same as the target protein that is directly regulated by the systems and methods as disclosed herein. In some examples, a population of cells (e.g., engineered immune cells) treated by the systems and methods disclosed herein can exhibit an expression or activity level of a cytokine that is higher than that in a comparable population of cells that has not been treated by the systems and methods, by at least or up to about 5%, at least or up to about 10%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 200%, at least or up to about 300%, at least or up to about 400%, at least or up to about 500%, at least or up to about 600%, at least or up to about 700%, at least or up to about 800%, at least or up to about 900%, at least or up to about 1,000%, at least or up to about 2,000%, at least or up to about 3,000%, at least or up to about 4,000%, or at least or up to about 5,000%.

In some cases, the cytokine production as disclosed herein can be measured upon at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, at least or up to about 4 weeks, at least or up to about 1 month, at least or up to about 2 months, at least or up to about 3 months, at least or up to about 4 months, at least or up to about 5 months, or at least or up to about 6 months after inducing the complexing as disclosed herein (e.g., that after activating the actuator moiety, or that after binding of the ligand to the ligand binding domain of the chimeric receptor).

In some cases, the cytokine production as disclosed herein can be ascertained (or can be measured) in vitro, ex vivo, or in vivo.

In some cases, the change (e.g., increase, decrease) in the expression or activity level of the cytokine (e.g., an endogenous cytokine) induced upon regulating expression or activity level of the target protein, as disclosed herein, can occur (or can be observed) in vitro, ex vivo, or in vivo.

In some cases, the change in the expression or activity of the target protein as provided herein can enhance differentiation of T cells (e.g., naïve T cells expressing the chimeric receptor as disclosed herein) into an immune cell sub-type (e.g., T helper (Th) cells expressing the chimeric receptor as disclosed herein). Non-limiting types of Th cells can include T follicular helper cells (Tfh cells, e.g., Bcl-6 positive), Th1 cells (e.g., T-bet positive), Th2 cells (e.g., Gata3 positive), Th17 cells (e.g., RAR-related orphan receptor gamma (such as RORY2) positive), and induced regulatory T (iTreg) cells (e.g., Foxp3 positive). In some examples, a population of engineered immune cells (e.g., naïve T cells) treated by the systems and methods disclosed herein can exhibit a proportion (e.g., percentage) of Th cells (e.g., Th1 cells) that is higher than that in a comparable population of immune cells that has not been treated by the systems and methods, by at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%.

In some cases, the proportion of immune cell sub-type(s) (e.g., Th1 cells) as disclosed herein can be measured upon at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, at least or up to about 4 weeks, at least or up to about 1 month, at least or up to about 2 months, at least or up to about 3 months, at least or up to about 4 months, at least or up to about 5 months, or at least or up to about 6 months after inducing the complexing as disclosed herein (e.g., that after activating the actuator moiety, or that after binding of the ligand to the ligand binding domain of the chimeric receptor).

In some cases, the proportion of immune cell sub-type(s) (e.g., Th1 cells) as disclosed herein can be ascertained (or can be measured) in vitro, ex vivo, or in vivo.

In some cases, the cytokine (e.g., an endogenous cytokine) can comprise IFN. In some cases, the cytokine can be selected from the group consisting of IFN-α (alpha), IFN-β (beta), IFN-κ (kappa), IFN-δ (delta), IFN-ε (epsilon), IFN-τ (tau), IFN-ω (omega), IFN-ζ (zeta), IFN-γ (gamma), and IFN-λ (lambda). In some cases, the cytokine can comprise IFN-γ (gamma). In some cases, upon regulating expression or activity level of the target protein in the cell, as disclosed herein, the cell can be effected to exhibit an increase in the expression or activity level of IFN (e.g., IFN-γ).

In some cases, the cytokine (e.g., an endogenous cytokine) can comprise a TNF protein. In some cases, the cytokine can be selected from the group consisting of TNFβ, TNFα, TNFγ, CD252 (OX40 ligand), CD154 (CD40 ligand), CD178 (Fas ligand), CD70 (CD27 ligand), CD153 (CD30 ligand), 4-1 BBL (CD137 ligand), CD253 (TRAIL), CD254 (RANKL), APO-3L (TWEAK), CD256 (APRIL), CD257 (BAFF), CD258 (LIGHT), TL1 (VEGI), GITRL (TNFSF18), and Ectodysplasin A. In some cases, the cytokine can comprise TNFα. In some cases, upon regulating expression or activity level of the target protein in the cell, as disclosed herein, the cell can be effected to exhibit an increase in the expression or activity level of TNF (e.g., TNFα).

In some cases, the cytokine (e.g., an endogenous cytokine) can comprise IL. The IL c cytokine In some cases, the IL can comprise one or more members selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36. In some cases, the cytokine can be IL-2. In some cases, the cell can be effected to exhibit a decrease in the expression or activity level of the IL.

In some cases, the change in the expression or activity of the target protein as provided herein can promote enhanced cytotoxicity of the engineered immune cell against a population of target cells.

In some cases, the enhanced cytotoxicity against the population of target cells can be ascertained by at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95% decrease in the size of the population of target cells.

In some cases, the enhanced cytotoxicity against the population of target cells as disclosed herein can be measured upon at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, at least or up to about 4 weeks, at least or up to about 1 month, at least or up to about 2 months, at least or up to about 3 months, at least or up to about 4 months, at least or up to about 5 months, or at least or up to about 6 months after inducing the complexing as disclosed herein (e.g., that after activating the actuator moiety, or that after binding of the ligand to the ligand binding domain of the chimeric receptor).

In some cases, the enhanced cytotoxicity against the population of target cells as disclosed herein can occur (or can be observed) in vitro, ex vivo, or in vivo.

In some examples, the enhanced cytotoxicity against the population of target cells as disclosed herein can be ascertained by measuring a size of a tumor (comprising a population of target cells, such as cancer cells) in a subject, e.g., subsequent to administering a cell comprising the system of the present disclosure to the subject. In some cases, a size of a tumor (e.g., a solid tumor) of the subject can be reduced by at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95% upon administration of the cell comprising the system of the present disclosure.

In some cases, the reduction in the size of the tumor can occur (or can be observed) upon at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, at least or up to about 4 weeks, at least or up to about 1 month, at least or up to about 2 months, at least or up to about 3 months, at least or up to about 4 months, at least or up to about 5 months, or at least or up to about 6 months after inducing the complexing as disclosed herein (e.g., that after activating the actuator moiety, or that after binding of the ligand to the ligand binding domain of the chimeric receptor).

In some cases, the actuator moiety can be heterologous to the cell. In some cases, the actuator moiety can be activatable for the complexing (e.g., forming a complex comprising the actuator moiety and the target polynucleotide sequence, as disclosed herein) upon exposure of the cell to an external stimulus (e.g., an extracellular ligand, such as an antigen).

In some cases, activation of the actuator moiety can comprise a modification (e.g., a conformational change, a chemical modification) of the actuator moiety. In some cases, the activation of the actuator moiety can comprise release of the actuator moiety from a substrate (e.g., a polypeptide substrate). In such a case, the actuator moiety may not be activated when bound to the substrate.

In some cases, the system can comprise a chimeric receptor polypeptide (receptor) that undergoes a modification upon binding to a ligand. The actuator moiety can be activatable (e.g., to regulate expression or activity level of the target protein) upon binding between the ligand and the chimeric receptor polypeptide, and/or upon the receptor modification. In some cases, the receptor can comprise an antigen binding moiety capable of specifically binding to at least one ligand (e.g., at least 1, 2, 3, 4, 5, or more ligands). The antigen binding moiety can be (i) monovalent or multivalent and (ii) monospecific or multispecific.

In some cases, the actuator moiety can be activated in absence of a signaling pathway involving one or more transcription factors (e.g., endogenous transcription factor(s)). Alternatively, the actuator moiety can be activated via a signaling pathway involving one or more transcription factors (e.g., endogenous transcription factor(s)).

In some cases, the target polynucleotide sequence as disclosed herein can be an endogenous gene. Alternatively or in addition to, the target polynucleotide sequence can be or a heterologous gene encoding the target protein (e.g., endogenous or heterologous protein). For example, the heterologous gene can comprise a natural amino acid sequence of the endogenous protein.

In some cases, the actuator moiety (e.g., an actuator moiety that is a part of a gene modulating polypeptide or GMP) as disclosed herein can be heterologous to the cell. The GMP can be a substrate, and activation of the actuator moiety can comprise release of the actuator moiety from the GMP upon the receptor modification, as disclosed herein. The GMP can be a part of a lager protein (e.g., a receptor polypeptide or an adaptor polypeptide as disclosed herein).

The target polynucleotide sequence as disclosed herein can comprise at least a portion of a transcription start site (TSS) of a target gene encoding the target protein. The target polynucleotide sequence can comprise at least about 5%, 10%, 15%, 20%, 30%, 40%, 50%, 60%, 70%, 75%, 80%, 85%, 90%, 95%, 96%, 97%, 98%, 99%, or 100% of the TSS of the target gene. The target polynucleotide sequence can comprise at most about 100%, 99%, 98%, 87%, 96%, 95%, 90%, 85%, 80%, 75%, 70%, 65%, 60%, 55%, 50%, 40%, 30%, 20%, 15%, 10%, 5%, or less of the TSS of the target gene. Alternatively of in addition to, the target polynucleotide sequence can be at most about 20,000 bases, 10,000 bases, 9,000 bases, 8,000 bases, 7,000 bases, 6,000 bases, 5,000 bases, 4,000 bases, 3,000 bases, 2,500 bases, 2,000 bases, 1,900 bases, 1,800 bases, 1,700 bases, 1,600 bases, 1,500 bases, 1,400 bases, 1,300 bases, 1,200 bases, 1,100 bases, 1,000 bases, 900 bases, 800 bases, 700 bases, 600 bases, 500 bases, 450 bases, 400 bases, 350 bases, 300 bases, 250 bases, 200 bases, 150 bases, 100 bases, or less away from the TSS of the target gene (e.g., at most about a disclosed number of bases upstream or downstream of a central nucleobase of the TSS of the target gene). At least a portion of the target polynucleotide sequence can be downstream of the TSS of the target gene. Alternatively or in addition to, at least a portion of the target polynucleotide sequence can be upstream of the TSS of the target gene. In some examples, a plurality of target polynucleotide sequences of the target gene can be utilized (e.g., complexed with the actuator moiety as disclosed herein) by the systems and methods disclosed herein, and the plurality of target polynucleotide sequences may comprise one or more members (e.g., 1 member, 2 members, or all 3 members) selected from the group consisting of (1) a target polynucleotide sequence that is at least partially downstream of the TSS of the target gene, (2) a target polynucleotide sequence that is at least partially upstream of the TSS of the target gene, and (3) the TSS of the target gene.

In some cases, a distance between the target polynucleotide sequence (e.g., a central nucleobase of the target polynucleotide sequence) and the TSS of the target gene (e.g., a central nucleobase of the TSS) can be about 1 base to about 10,000 bases. The distance between the target polynucleotide sequence and the TSS of the target gene can be at least about 1 base. The distance between the target polynucleotide sequence and the TSS of the target gene can be at most about 10,000 bases. The distance between the target polynucleotide sequence and the TSS of the target gene can be about 10,000 bases to about 9,000 bases, about 10,000 bases to about 8,000 bases, about 10,000 bases to about 7,000 bases, about 10,000 bases to about 6,000 bases, about 10,000 bases to about 5,000 bases, about 10,000 bases to about 4,000 bases, about 10,000 bases to about 3,000 bases, about 10,000 bases to about 2,000 bases, about 10,000 bases to about 1,000 bases, about 10,000 bases to about 500 bases, about 10,000 bases to about 1 base, about 9,000 bases to about 8,000 bases, about 9,000 bases to about 7,000 bases, about 9,000 bases to about 6,000 bases, about 9,000 bases to about 5,000 bases, about 9,000 bases to about 4,000 bases, about 9,000 bases to about 3,000 bases, about 9,000 bases to about 2,000 bases, about 9,000 bases to about 1,000 bases, about 9,000 bases to about 500 bases, about 9,000 bases to about 1 base, about 8,000 bases to about 7,000 bases, about 8,000 bases to about 6,000 bases, about 8,000 bases to about 5,000 bases, about 8,000 bases to about 4,000 bases, about 8,000 bases to about 3,000 bases, about 8,000 bases to about 2,000 bases, about 8,000 bases to about 1,000 bases, about 8,000 bases to about 500 bases, about 8,000 bases to about 1 base, about 7,000 bases to about 6,000 bases, about 7,000 bases to about 5,000 bases, about 7,000 bases to about 4,000 bases, about 7,000 bases to about 3,000 bases, about 7,000 bases to about 2,000 bases, about 7,000 bases to about 1,000 bases, about 7,000 bases to about 500 bases, about 7,000 bases to about 1 base, about 6,000 bases to about 5,000 bases, about 6,000 bases to about 4,000 bases, about 6,000 bases to about 3,000 bases, about 6,000 bases to about 2,000 bases, about 6,000 bases to about 1,000 bases, about 6,000 bases to about 500 bases, about 6,000 bases to about 1 base, about 5,000 bases to about 4,000 bases, about 5,000 bases to about 3,000 bases, about 5,000 bases to about 2,000 bases, about 5,000 bases to about 1,000 bases, about 5,000 bases to about 500 bases, about 5,000 bases to about 1 base, about 4,000 bases to about 3,000 bases, about 4,000 bases to about 2,000 bases, about 4,000 bases to about 1,000 bases, about 4,000 bases to about 500 bases, about 4,000 bases to about 1 base, about 3,000 bases to about 2,000 bases, about 3,000 bases to about 1,000 bases, about 3,000 bases to about 500 bases, about 3,000 bases to about 1 base, about 2,000 bases to about 1,000 bases, about 2,000 bases to about 500 bases, about 2,000 bases to about 1 base, about 1,000 bases to about 500 bases, about 1,000 bases to about 1 base, or about 500 bases to about 1 base. The distance between the target polynucleotide sequence and the TSS of the target gene can be about 10,000 bases, about 9,000 bases, about 8,000 bases, about 7,000 bases, about 6,000 bases, about 5,000 bases, about 4,000 bases, about 3,000 bases, about 2,000 bases, about 1,000 bases, about 500 bases, or about 1 base.

In some cases, a distance between the target polynucleotide sequence (e.g., a central nucleobase of the target polynucleotide sequence) and the TSS of the target gene (e.g., a central nucleobase of the TSS) can be about 1 base to about 5,000 bases. The distance between the target polynucleotide sequence and the TSS of the target gene can be at least about 1 base. The distance between the target polynucleotide sequence and the TSS of the target gene can be at most about 5,000 bases. The distance between the target polynucleotide sequence and the TSS of the target gene can be about 5,000 bases to about 4,500 bases, about 5,000 bases to about 4,000 bases, about 5,000 bases to about 3,500 bases, about 5,000 bases to about 3,000 bases, about 5,000 bases to about 2,500 bases, about 5,000 bases to about 2,000 bases, about 5,000 bases to about 1,500 bases, about 5,000 bases to about 1,000 bases, about 5,000 bases to about 500 bases, about 5,000 bases to about 100 bases, about 5,000 bases to about 1 base, about 4,500 bases to about 4,000 bases, about 4,500 bases to about 3,500 bases, about 4,500 bases to about 3,000 bases, about 4,500 bases to about 2,500 bases, about 4,500 bases to about 2,000 bases, about 4,500 bases to about 1,500 bases, about 4,500 bases to about 1,000 bases, about 4,500 bases to about 500 bases, about 4,500 bases to about 100 bases, about 4,500 bases to about 1 base, about 4,000 bases to about 3,500 bases, about 4,000 bases to about 3,000 bases, about 4,000 bases to about 2,500 bases, about 4,000 bases to about 2,000 bases, about 4,000 bases to about 1,500 bases, about 4,000 bases to about 1,000 bases, about 4,000 bases to about 500 bases, about 4,000 bases to about 100 bases, about 4,000 bases to about 1 base, about 3,500 bases to about 3,000 bases, about 3,500 bases to about 2,500 bases, about 3,500 bases to about 2,000 bases, about 3,500 bases to about 1,500 bases, about 3,500 bases to about 1,000 bases, about 3,500 bases to about 500 bases, about 3,500 bases to about 100 bases, about 3,500 bases to about 1 base, about 3,000 bases to about 2,500 bases, about 3,000 bases to about 2,000 bases, about 3,000 bases to about 1,500 bases, about 3,000 bases to about 1,000 bases, about 3,000 bases to about 500 bases, about 3,000 bases to about 100 bases, about 3,000 bases to about 1 base, about 2,500 bases to about 2,000 bases, about 2,500 bases to about 1,500 bases, about 2,500 bases to about 1,000 bases, about 2,500 bases to about 500 bases, about 2,500 bases to about 100 bases, about 2,500 bases to about 1 base, about 2,000 bases to about 1,500 bases, about 2,000 bases to about 1,000 bases, about 2,000 bases to about 500 bases, about 2,000 bases to about 100 bases, about 2,000 bases to about 1 base, about 1,500 bases to about 1,000 bases, about 1,500 bases to about 500 bases, about 1,500 bases to about 100 bases, about 1,500 bases to about 1 base, about 1,000 bases to about 500 bases, about 1,000 bases to about 100 bases, about 1,000 bases to about 1 base, about 500 bases to about 100 bases, about 500 bases to about 1 base, or about 100 bases to about 1 base. The distance between the target polynucleotide sequence and the TSS of the target gene can be about 5,000 bases, about 4,500 bases, about 4,000 bases, about 3,500 bases, about 3,000 bases, about 2,500 bases, about 2,000 bases, about 1,500 bases, about 1,000 bases, about 500 bases, about 100 bases, or about 1 base.

In some cases, a distance between the target polynucleotide sequence (e.g., a central nucleobase of the target polynucleotide sequence) and the TSS of the target gene (e.g., a central nucleobase of the TSS) can be about 1 base to about 2,000 bases. The distance between the target polynucleotide sequence and the TSS of the target gene can be at least about 1 base. The distance between the target polynucleotide sequence and the TSS of the target gene can be at most about 2,000 bases. The distance between the target polynucleotide sequence and the TSS of the target gene can be about 2,000 bases to about 1,800 bases, about 2,000 bases to about 1,600 bases, about 2,000 bases to about 1,400 bases, about 2,000 bases to about 1,200 bases, about 2,000 bases to about 1,000 bases, about 2,000 bases to about 800 bases, about 2,000 bases to about 600 bases, about 2,000 bases to about 400 bases, about 2,000 bases to about 200 bases, about 2,000 bases to about 1 base, about 1,800 bases to about 1,600 bases, about 1,800 bases to about 1,400 bases, about 1,800 bases to about 1,200 bases, about 1,800 bases to about 1,000 bases, about 1,800 bases to about 800 bases, about 1,800 bases to about 600 bases, about 1,800 bases to about 400 bases, about 1,800 bases to about 200 bases, about 1,800 bases to about 1 base, about 1,600 bases to about 1,400 bases, about 1,600 bases to about 1,200 bases, about 1,600 bases to about 1,000 bases, about 1,600 bases to about 800 bases, about 1,600 bases to about 600 bases, about 1,600 bases to about 400 bases, about 1,600 bases to about 200 bases, about 1,600 bases to about 1 base, about 1,400 bases to about 1,200 bases, about 1,400 bases to about 1,000 bases, about 1,400 bases to about 800 bases, about 1,400 bases to about 600 bases, about 1,400 bases to about 400 bases, about 1,400 bases to about 200 bases, about 1,400 bases to about 1 base, about 1,200 bases to about 1,000 bases, about 1,200 bases to about 800 bases, about 1,200 bases to about 600 bases, about 1,200 bases to about 400 bases, about 1,200 bases to about 200 bases, about 1,200 bases to about 1 base, about 1,000 bases to about 800 bases, about 1,000 bases to about 600 bases, about 1,000 bases to about 400 bases, about 1,000 bases to about 200 bases, about 1,000 bases to about 1 base, about 800 bases to about 600 bases, about 800 bases to about 400 bases, about 800 bases to about 200 bases, about 800 bases to about 1 base, about 600 bases to about 400 bases, about 600 bases to about 200 bases, about 600 bases to about 1 base, about 400 bases to about 200 bases, about 400 bases to about 1 base, or about 200 bases to about 1 base. The distance between the target polynucleotide sequence and the TSS of the target gene can be about 2,000 bases, about 1,800 bases, about 1,600 bases, about 1,400 bases, about 1,200 bases, about 1,000 bases, about 800 bases, about 600 bases, about 400 bases, about 200 bases, or about 1 base.

In some cases, a distance between the target polynucleotide sequence (e.g., a central nucleobase of the target polynucleotide sequence) and the TSS of the target gene (e.g., a central nucleobase of the TSS) can be about 1 base to about 1,000 bases. The distance between the target polynucleotide sequence and the TSS of the target gene can be at least about 1 base. The distance between the target polynucleotide sequence and the TSS of the target gene can be at most about 1,000 bases. The distance between the target polynucleotide sequence and the TSS of the target gene can be about 1,000 bases to about 900 bases, about 1,000 bases to about 800 bases, about 1,000 bases to about 700 bases, about 1,000 bases to about 600 bases, about 1,000 bases to about 500 bases, about 1,000 bases to about 400 bases, about 1,000 bases to about 300 bases, about 1,000 bases to about 200 bases, about 1,000 bases to about 100 bases, about 1,000 bases to about 1 base, about 900 bases to about 800 bases, about 900 bases to about 700 bases, about 900 bases to about 600 bases, about 900 bases to about 500 bases, about 900 bases to about 400 bases, about 900 bases to about 300 bases, about 900 bases to about 200 bases, about 900 bases to about 100 bases, about 900 bases to about 1 base, about 800 bases to about 700 bases, about 800 bases to about 600 bases, about 800 bases to about 500 bases, about 800 bases to about 400 bases, about 800 bases to about 300 bases, about 800 bases to about 200 bases, about 800 bases to about 100 bases, about 800 bases to about 1 base, about 700 bases to about 600 bases, about 700 bases to about 500 bases, about 700 bases to about 400 bases, about 700 bases to about 300 bases, about 700 bases to about 200 bases, about 700 bases to about 100 bases, about 700 bases to about 1 base, about 600 bases to about 500 bases, about 600 bases to about 400 bases, about 600 bases to about 300 bases, about 600 bases to about 200 bases, about 600 bases to about 100 bases, about 600 bases to about 1 base, about 500 bases to about 400 bases, about 500 bases to about 300 bases, about 500 bases to about 200 bases, about 500 bases to about 100 bases, about 500 bases to about 1 base, about 400 bases to about 300 bases, about 400 bases to about 200 bases, about 400 bases to about 100 bases, about 400 bases to about 1 base, about 300 bases to about 200 bases, about 300 bases to about 100 bases, about 300 bases to about 1 base, about 200 bases to about 100 bases, about 200 bases to about 1 base, or about 100 bases to about 1 base. The distance between the target polynucleotide sequence and the TSS of the target gene can be about 1,000 bases, about 900 bases, about 800 bases, about 700 bases, about 600 bases, about 500 bases, about 400 bases, about 300 bases, about 200 bases, about 100 bases, or about 1 base.

In some cases, a distance between the target polynucleotide sequence (e.g., a central nucleobase of the target polynucleotide sequence) and the TSS of the target gene (e.g., a central nucleobase of the TSS) can be about 1 base to about 500 bases. The distance between the target polynucleotide sequence and the TSS of the target gene can be at least about 1 base. The distance between the target polynucleotide sequence and the TSS of the target gene can be at most about 500 bases. The distance between the target polynucleotide sequence and the TSS of the target gene can be about 500 bases to about 450 bases, about 500 bases to about 400 bases, about 500 bases to about 350 bases, about 500 bases to about 300 bases, about 500 bases to about 250 bases, about 500 bases to about 200 bases, about 500 bases to about 150 bases, about 500 bases to about 100 bases, about 500 bases to about 50 bases, about 500 bases to about 1 base, about 450 bases to about 400 bases, about 450 bases to about 350 bases, about 450 bases to about 300 bases, about 450 bases to about 250 bases, about 450 bases to about 200 bases, about 450 bases to about 150 bases, about 450 bases to about 100 bases, about 450 bases to about 50 bases, about 450 bases to about 1 base, about 400 bases to about 350 bases, about 400 bases to about 300 bases, about 400 bases to about 250 bases, about 400 bases to about 200 bases, about 400 bases to about 150 bases, about 400 bases to about 100 bases, about 400 bases to about 50 bases, about 400 bases to about 1 base, about 350 bases to about 300 bases, about 350 bases to about 250 bases, about 350 bases to about 200 bases, about 350 bases to about 150 bases, about 350 bases to about 100 bases, about 350 bases to about 50 bases, about 350 bases to about 1 base, about 300 bases to about 250 bases, about 300 bases to about 200 bases, about 300 bases to about 150 bases, about 300 bases to about 100 bases, about 300 bases to about 50 bases, about 300 bases to about 1 base, about 250 bases to about 200 bases, about 250 bases to about 150 bases, about 250 bases to about 100 bases, about 250 bases to about 50 bases, about 250 bases to about 1 base, about 200 bases to about 150 bases, about 200 bases to about 100 bases, about 200 bases to about 50 bases, about 200 bases to about 1 base, about 150 bases to about 100 bases, about 150 bases to about 50 bases, about 150 bases to about 1 base, about 100 bases to about 50 bases, about 100 bases to about 1 base, or about 50 bases to about 1 base. The distance between the target polynucleotide sequence and the TSS of the target gene can be about 500 bases, about 450 bases, about 400 bases, about 350 bases, about 300 bases, about 250 bases, about 200 bases, about 150 bases, about 100 bases, about 50 bases, or about 1 base.

In some cases, a distance between the target polynucleotide sequence (e.g., a central nucleobase of the target polynucleotide sequence) and the TSS of the target gene (e.g., a central nucleobase of the TSS) can be about 1 base to about 250 bases. The distance between the target polynucleotide sequence and the TSS of the target gene can be at least about 1 base. The distance between the target polynucleotide sequence and the TSS of the target gene can be at most about 250 bases. The distance between the target polynucleotide sequence and the TSS of the target gene can be about 250 bases to about 225 bases, about 250 bases to about 200 bases, about 250 bases to about 175 bases, about 250 bases to about 150 bases, about 250 bases to about 125 bases, about 250 bases to about 100 bases, about 250 bases to about 75 bases, about 250 bases to about 50 bases, about 250 bases to about 25 bases, about 250 bases to about 1 base, about 225 bases to about 200 bases, about 225 bases to about 175 bases, about 225 bases to about 150 bases, about 225 bases to about 125 bases, about 225 bases to about 100 bases, about 225 bases to about 75 bases, about 225 bases to about 50 bases, about 225 bases to about 25 bases, about 225 bases to about 1 base, about 200 bases to about 175 bases, about 200 bases to about 150 bases, about 200 bases to about 125 bases, about 200 bases to about 100 bases, about 200 bases to about 75 bases, about 200 bases to about 50 bases, about 200 bases to about 25 bases, about 200 bases to about 1 base, about 175 bases to about 150 bases, about 175 bases to about 125 bases, about 175 bases to about 100 bases, about 175 bases to about 75 bases, about 175 bases to about 50 bases, about 175 bases to about 25 bases, about 175 bases to about 1 base, about 150 bases to about 125 bases, about 150 bases to about 100 bases, about 150 bases to about 75 bases, about 150 bases to about 50 bases, about 150 bases to about 25 bases, about 150 bases to about 1 base, about 125 bases to about 100 bases, about 125 bases to about 75 bases, about 125 bases to about 50 bases, about 125 bases to about 25 bases, about 125 bases to about 1 base, about 100 bases to about 75 bases, about 100 bases to about 50 bases, about 100 bases to about 25 bases, about 100 bases to about 1 base, about 75 bases to about 50 bases, about 75 bases to about 25 bases, about 75 bases to about 1 base, about 50 bases to about 25 bases, about 50 bases to about 1 base, or about 25 bases to about 1 base. The distance between the target polynucleotide sequence and the TSS of the target gene can be about 250 bases, about 225 bases, about 200 bases, about 175 bases, about 150 bases, about 125 bases, about 100 bases, about 75 bases, about 50 bases, about 25 bases, or about 1 base.

In some cases, the target protein can be a secretory protein. In some cases, the target protein may not be a secretory protein.

In some cases, the cell can be effected to exhibit at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 200%, at least or up to about 300%, at least or up to about 400%, at least or up to about 500%, at least or up to about 600%, at least or up to about 700%, at least or up to about 800%, at least or up to about 900%, at least or up to about 1,000%, at least or up to about 2,000%, at least or up to about 3,000%, at least or up to about 4,000%, or at least or up to about 5,000% change in the expression or activity level of the target protein as compared to a comparable cell that has not been treated with the systems and methods disclosed herein.

In some cases, the cell can be effected to exhibit at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 200%, at least or up to about 300%, at least or up to about 400%, at least or up to about 500%, at least or up to about 600%, at least or up to about 700%, at least or up to about 800%, at least or up to about 900%, at least or up to about 1,000%, at least or up to about 2,000%, at least or up to about 3,000%, at least or up to about 4,000%, or at least or up to about 5,000% increase in the expression or activity level of the target protein (as compared to the control cell.

In some cases, the cell can be effected to exhibit at least or up to about 20%, at least or up to about 30%, at least or up to about 40%, at least or up to about 50%, at least or up to about 60%, at least or up to about 70%, at least or up to about 80%, at least or up to about 90%, at least or up to about 95%, decrease in the expression or activity level of the target protein as compared to the control cell.

In some cases, the change (e.g., increase, decrease) in the expression or activity level of the target protein as compared to the control cell can be observed upon at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 2 weeks, at least or up to about 3 weeks, or at least or up to about 4 weeks of the activation of the actuator moiety (or upon receptor modification, or upon binding of the ligand to the ligand binding domain of the chimeric receptor) as disclosed herein.

In some cases, the actuator moiety can comprise a nucleic acid-guided actuator moiety. In some cases, the system can further comprise a guide nucleic acid that complexes with the actuator moiety. In some cases, the system further comprises two or more guide nucleic acids (e.g., at least 2, 3, 4, 5, 6, 7, 8, 9, 10, or more guide nucleic acids) having complementarity to different target polynucleotide sequences (e.g., different portions of the target gene encoding the target protein). In some cases, a guide nucleic acid as disclosed herein can comprise a guide ribonucleic acid (RNA). In some examples, the cell as disclosed herein can comprise (1) a first guide nucleic acid (e.g., a first guide RNA) capable of binding a first target polynucleotide sequence that is operatively coupled to the target protein and (2) a second guide nucleic acid (e.g., a second guide RNA) capable of binding a second target polynucleotide sequence that is operatively coupled to the target protein.

In some cases, the ligand as disclosed herein can be an antigen of diseased cells. In some cases, the population of target cells as disclosed herein can comprise diseased cells. In some cases, the diseased cells as disclosed herein can comprise cancer cells or tumor cells.

In some cases, the cell can be a hematopoietic stem cell (HSC). In some cases, the cell can be an immune cell (lymphocyte). In some cases, the immune cell can be selected from the group consisting of a T cell, an NK cell, a monocyte, an innate lymphocyte, a tumor-infiltrating lymphocyte, a macrophage, and a granulocyte.

In some cases, a control as disclosed herein can be a control cell without one or more members comprising (i) a functional chimeric receptor polypeptide, (ii) a functional actuator moiety, (iii) a functional guide nucleic acid sequence (e.g., a functional guide RNA) designed to target the target gene, (iv) a chimeric adaptor polypeptide operatively coupled to the chimeric receptor polypeptide (as discussed below). In some cases, a cell can utilize a guide nucleic acid sequence, and a control cell may comprise a control nucleic acid sequence that is not designed to complex with the target polynucleotide sequence. In some cases, a cell can utilize two different guide nucleic acid sequences, and a control cell may comprise none or only one of the two different guide nucleic acid sequences.

In some cases, the systems of the present disclosure can enhance an immune response in a subject. Non-limiting of enhancement of immune response can include increased CD4+ helper T cell activity and generation of cytolytic T cells. The enhancement of immune response can be assessed using a number of in vitro or in vivo measurements known to those skilled in the art, including, but not limited to, cytotoxic T lymphocyte assays, release of cytokines (e.g., IL-12, IL-2, or IFN-γ production). regression of tumors, survival of tumor bearing animals, antibody production, immune cell proliferation, expression of cell surface markers, and cytotoxicity.

In some cases, regulated expression and/or activity of a protein (e.g., endogenous cytokine) as disclosed herein can be ascertained by a number of methods, including, but are not limited to, (i) phosphorylation of a downstream signaling protein (e.g., (a) TYK2, JAK2, or STAT4 for IL-12 signaling; (b) JAK1, JAK2, STAT1, STAT2, or STAT3 for IL-21 signaling; (c) JAK1, JAK2, or STAT3 for IFN-γ signaling; (d) PI3K, Akt, IκB kinase, STAT5 for TNFα signaling, etc.) or (ii) expression of a downstream gene (e.g., IFN-γ or TNFα) via Western blotting or polymerase chain reaction (PCR) techniques.

In one aspect, the present disclosure provides a population of cells comprising any one of the systems disclosed herein. In some cases, the population of cells can comprise engineered immune cells. In some cases, the engineered immune cells comprise engineered T cells. In some cases, the engineered immune cells comprise engineered NK cells.

### III. Chimeric Receptor Polypeptide

In some cases, the chimeric receptor polypeptide (receptor) as disclosed herein can be operatively coupled to a chimeric adaptor polypeptide (adaptor). In some cases, the receptor and the adaptor can be configured to form a complex (e.g., a signaling complex) upon binding of the ligand to the receptor (e.g., upon contacting the cell comprising the receptor with the ligand) and/or upon the receptor modification. The adaptor can be a transmembrane protein. Alternatively, the adaptor can be an intracellular protein. In some cases, the adaptor can be signaling protein of the receptor signaling pathway that is recruited towards the receptor upon the receptor modification.

In some cases, the complexation of the receptor and the adaptor can be direct and/or indirect. In a direct complexation, one of the receptor and the adaptor can be configured to directly bind (e.g., via covalent and/or non-covalent interactions) to the other of the receptor and the adaptor. In some examples, one of the receptor and the adaptor can comprise a binding domain (e.g., a polypeptide sequence) configured to bind to at least a portion (e.g., an intracellular portion) of the other of the receptor and the adaptor. In an indirect complexation, the receptor and the adaptor can be configured to be brought closer to each other (e.g., one is recruited towards the other) without any direct binding upon the receptor modification, relative to without the receptor modification. In some examples, the receptor can comprise a chimeric antigen receptor (CAR) or a modified immune cell receptor (e.g., a modified T cell receptor or “TCR”), and the adaptor can comprise at least a portion of Linker for activation of T cells (LAT) that is recruited as part of a signaling cascade of the receptor upon the receptor modification.

In some cases, one of the receptor and the adaptor can comprise a gene modulating polypeptide comprising the actuator moiety linked to a cleavage recognition site, and the other of the receptor and the adaptor can comprise a cleavage moiety configured to cleave the cleavage recognition site to release the actuator moiety from the GMP. In some examples, the cleaving of the cleavage recognition site by the cleavage moiety can occur upon a direct complexation between the receptor and the adaptor. In some examples, the cleaving of the cleavage recognition site by the cleavage moiety can occur upon an indirect complexation between the receptor and the adaptor. Upon the receptor confirmation, the receptor and the adaptor can be recruited towards each other, such that the cleavage moiety can cleave the actuator moiety from the GMP, thereby to activate the actuator moiety to regulate expression or activity of the endogenous protein (e.g., endogenous cytokine), as disclosed herein.

In some cases, the chimeric receptor polypeptide (receptor) as disclosed herein can be operatively coupled to a first chimeric adaptor polypeptide (a first adaptor) and a second chimeric adaptor polypeptide (a second adaptor). In some cases, the first adaptor and the second adaptor can be signaling proteins of the receptor signaling pathway that are recruited towards the receptor or towards another signaling protein of the receptor signaling pathway upon the receptor modification. In some examples, the first adaptor and the second adaptor can be recruited towards each other upon the receptor modification. As disclosed herein, the first adaptor and the second adaptor can form a complex via a direct binding. Alternatively, the first adaptor and the second adaptor can form a complex via an indirect binding (e.g., in the vicinity of each other). In some cases, a first adaptor can comprise the GMP (comprising the actuator moiety linked to the cleavage recognition site) and a second adaptor can comprise the cleavage moiety, as disclosed herein. Upon the receptor confirmation, the first adaptor and the second adaptor can be recruited towards each other, such that the cleavage moiety can cleave the actuator moiety from the GMP, thereby to activate the actuator moiety to regulate expression or activity of the endogenous protein (e.g., endogenous cytokine).

In some cases, one of the first and second adaptors can comprise a gene modulating polypeptide comprising the actuator moiety linked to a cleavage recognition site, and the other of the first and second adaptors can comprise a cleavage moiety configured to cleave the cleavage recognition site to release the actuator moiety from the GMP. In some examples, the cleaving of the cleavage recognition site by the cleavage moiety can occur upon a direct complexation between the first and second adaptors. In some examples, the cleaving of the cleavage recognition site by the cleavage moiety can occur upon an indirect complexation between the first and second adaptors.

In some cases, the receptor as disclosed herein can undergo a receptor modification including a conformational change or chemical modification (e.g., phosphorylation or dephosphorylation) upon binding to the ligand.

FIGS. 1A-1D schematically illustrate the release of an actuator moiety from a GMP. FIG. 1A shows the binding of an antigen to a transmembrane chimeric receptor polypeptide. The transmembrane chimeric receptor polypeptide comprises an extracellular region having an antigen interacting domain 101 and an intracellular region comprising a GMP. The GMP includes an actuator moiety 102a linked to a cleavage recognition site 102b. In response to antigen binding, the receptor is modified by phosphorylation 103 in the intracellular region of the receptor (FIG. 1B). Following receptor modification (e.g., phosphorylation), an adaptor protein comprising a receptor binding moiety is recruited to the receptor as shown in FIG. 1C. The receptor comprises a cleavage moiety 104; the cleavage moiety may be complexed with the adaptor or linked, for example by a peptide bond and/or peptide linker, to the receptor binding moiety. When in proximity to the cleavage recognition site, the cleavage moiety can cleave the recognition site to release the actuator moiety from the GMP as shown in FIG. 1D. Upon release, the actuator moiety can enter the nucleus to regulate the expression and/or activity of a target gene (e.g., target protein as disclosed herein) or edit a nucleic acid sequence. FIGS. 1E-1H show an analogous system wherein receptor modification comprises a conformational change. In some embodiments, the adaptor protein is tethered to the membrane (e.g., as a membrane bound protein).

FIGS. 2A-2D illustrate schematically the release of an actuator moiety from a GMP. FIG. 2A shows the binding of an antigen to a transmembrane chimeric receptor polypeptide. The transmembrane chimeric receptor polypeptide comprises an extracellular region having an antigen interacting domain 205 and an intracellular region comprising a cleavage moiety 206. The cleavage moiety can be complexed with the receptor or linked, for example by a peptide bond and/or peptide linker, to the receptor. The GMP forms a portion of the chimeric adaptor polypeptide. The GMP, linked to a receptor binding moiety 201, includes an actuator moiety 202a linked to a cleavage recognition site 202b. In response to antigen binding, the receptor is modified by phosphorylation 203 in the intracellular region of the receptor (FIG. 2B). Following receptor modification (e.g., phosphorylation), the chimeric adaptor polypeptide is recruited to the receptor as shown in FIG. 3C. The receptor comprises a cleavage moiety 206. When in proximity to the cleavage recognition site, the cleavage moiety can cleave the recognition site to release the actuator moiety from the GMP as shown in FIG. 2D. Upon release, the actuator moiety can enter the nucleus to regulate the expression and/or activity of a target gene or edit a nucleic acid sequence. FIGS. 2E-2H show an analogous system wherein receptor modification comprises a conformational change. In some embodiments, the chimeric adaptor protein is tethered to the membrane (e.g., as a membrane bound protein).

FIGS. 3A-D illustrate schematically the release of an actuator moiety from a GMP. FIG. 3A shows the binding of an antigen to a transmembrane chimeric receptor polypeptide. The transmembrane chimeric receptor polypeptide comprises an extracellular region having an antigen interacting domain 305 and an intracellular region. The GMP, comprising an actuator moiety linked to a cleavage recognition site, forms a portion of a chimeric adaptor polypeptide. The cleavage recognition site 302b is flanked by the receptor binding moiety 301 and the actuator moiety 302a. In response to antigen binding, the receptor is modified by phosphorylation 303 in the intracellular region (FIG. 3B). Following receptor modification (e.g., phosphorylation), the chimeric adaptor polypeptide is recruited to the receptor as shown in FIG. 3B. A second adaptor polypeptide 307 comprising a cleavage moiety 306 is also recruited to the modified receptor (FIG. 3C). The cleavage moiety may be complexed with the second adaptor polypeptide or linked, for example by a peptide bond and/or peptide linker, to the adaptor. When in proximity to the cleavage recognition site, the cleavage moiety can cleave the recognition site to release the actuator moiety from the GMP as shown in FIG. 3D. Upon release, the actuator moiety can enter the nucleus to regulate the expression and/or activity of a target gene or edit a nucleic acid sequence. FIGS. 3E-H show an analogous system wherein receptor modification comprises a conformational change. In some embodiments, the chimeric adaptor polypeptide is tethered to the membrane (e.g., as a membrane bound protein). In some embodiments, the second adaptor polypeptide is tethered to the membrane (e.g., as a membrane bound protein).

In some cases, the chimeric receptor polypeptide (receptor) can comprise a ligand binding domain, a transmembrane domain, and a signaling domain. The signaling domain may activate a signaling pathway of the cell upon binding of a ligand to the ligand binding domain. The cell can further comprise an expression cassette comprising a polynucleotide sequence encoding an actuator moiety as disclosed herein (e.g., a GMP comprising the actuator moiety) placed under control of a promoter. The actuator moiety can comprise a heterologous endonuclease. The promoter can be activated to drive expression of the actuator moiety upon binding of the ligand to the ligand binding domain. The expressed actuator moiety can complex with a target gene encoding the endogenous protein (e.g., endogenous cytokine) as disclosed herein to regulate expression or activity of the endogenous protein. The promoter can comprise an endogenous promoter of the cell. The endogenous promoter can be activated upon binding of the ligand to the ligand binding domain of the receptor.

FIG. 4 illustrates an illustrative system comprising a transmembrane receptor useful for regulating expression of at least one target gene. Upon binding of a ligand with a chimeric receptor polypeptide (e.g., scFv-CAR), an intrinsic signal transduction pathway is activated, resulting in the recruitment of at least one cellular transcription factor (e.g., endogenous transcription factor) to the promoter region of an endogenous gene (a signature gene) at its natural locus. An actuator moiety coding sequence (e.g., a GMP coding sequence comprising an actuator moiety coding sequence) is integrated into the genome and is placed under the control of the promoter of the signature gene. Transcriptional activation of the promoter results in expression of the actuator moiety (e.g., comprising a dCas linked to a transcriptional activator (e.g., VPR) or a transcription repressor (e.g., KRAB). The expressed actuator moiety, upon complexing with a guide RNA (e.g., sgRNAa, sgRNAb) (e.g., constitutively or conditionally expressed), can regulate (activate or suppress) the expression of the endogenous protein as disclosed herein (e.g., Gene A such as IL-12A, Gene B such as IL-12B).

In some cases, the chimeric receptor polypeptide (receptor) as disclosed herein can be a chimeric antigen receptor (CAR) and/or a modified T cell receptor (TCR).

In some cases, a CAR as disclosed herein can be a first-, second-, third-, or fourth-generation CAR system, a functional variant thereof, or any combination thereof. First-generation CARs (e.g., CD19R or CD19CAR) include an antigen binding domain with specificity for a particular antigen (e.g., an antibody or antigen-binding fragment thereof such as an scFv, a Fab fragment, a VHH domain, or a VH domain of a heavy-chain only antibody), a transmembrane domain derived from an adaptive immune receptor (e.g., the transmembrane domain from the CD28 receptor), and a signaling domain derived from an adaptive immune receptor (e.g., one or more (e.g., three) ITAM domains derived from the intracellular region of the CD3 ζ receptor or FcεRIγ). Second-generation CARs modify the first-generation CAR by addition of a co-stimulatory domain to the intracellular signaling domain portion of the CAR (e.g., derived from co-stimulatory receptors that act alongside T-cell receptors such as CD28, CD137/4-1BB, and CD134/OX40), which abrogates the need for administration of a co-factor (e.g., IL-2) alongside a first-generation CAR. Third-generation CARs add multiple co-stimulatory domains to the intracellular signaling domain portion of the CAR (e.g., CD3ζ-CD28-OX40, or CD3ζ-CD28-41BB). Fourth-generation CARs modify second- or third-generation CARs by the addition of an activating cytokine (e.g., IL-23, or IL-27) to the intracellular signaling portion of the CAR (e.g., between one or more of the costimulatory domains and the CD3ζ ITAM domain) or under the control of a CAR-induced promoter (e.g., the NFAT/IL-2 minimal promoter).

### IV. Actuator Moiety

The actuator moiety (e.g., an actuator moiety that is a part of a GMP) as disclosed herein can be capable of editing (e.g., via insertion and/or deletion (indel), homology directed repair (HDR), non-homologous end joining (NHEJ)) the target gene, to regulate expression or activity of the target protein (e.g., endogenous protein(s)). Alternatively, the actuator moiety may not be capable of editing the target gene, but still exhibit the ability to complex with the target gene (e.g., deactivated or dead CRISPR/Cas protein, as provided herein).

The actuator moiety (e.g., an actuator moiety that is a part of a GMP) as disclosed herein can be operatively coupled to at least one effector domain. The at least one effector domain can be configured to regulate the expression or activity of the endogenous protein (e.g., endogenous cytokine), In some cases, the actuator moiety can be fused to at least one effector domain, to form a fusion moiety. In some cases, the actuator moiety can comprise a first coupling moiety (e.g., a polynucleotide) and the at least one effector domain can comprise a second coupling moiety (e.g., a second polynucleotide having complementarity to the first polynucleotide), such that the actuator moiety and the at least one effector domain can be coupled to one another. In some examples, the at least one effector domain can be a cleavage domain, an epigenetic modification domain, a transcriptional activation domain, or a transcriptional repressor domain, to regulate expression or activity of the endogenous protein (e.g., endogenous cytokine).

Non-limiting examples of a function of the at least one effector domain as disclosed herein can include methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, and demyristoylation activity.

Non-limiting examples of the at least one effector domain as disclosed herein can include methyltransferase, demethylase, dismutase, alkylation enzyme, depurination enzyme, oxidation enzyme, pyrimidine dimer forming enzyme, integrase, transposase, recombinase, polymerase, ligase, helicase, photolyase or glycosylase, acetyltransferase, deacetylase, kinase, phosphatase, ubiquitin ligase, deubiquitinating enzyme, adenylation enzyme, deadenylation enzyme, SUMOylating enzyme, deSUMOylating enzyme, ribosylation enzyme, deribosylation enzyme, myristoylation enzyme, remodeling enzyme, protease, oxidoreductase, transferase, hydrolase, lyase, isomerase, synthase, synthetase, and demyristoylation enzyme.

The actuator moiety as disclosed herein can comprise a nuclease, such as an endonuclease (e.g., Cas). The endonuclease can be heterologous to any of the cells disclosed herein.

The actuator moiety as disclosed herein can comprise a Cas endonuclease, zinc finger nuclease (ZFN), zinc finger associate gene regulation polypeptides, transcription activator-like effector nuclease (TALEN), transcription activator-like effector associated gene regulation polypeptides, meganuclease, natural master transcription factors, epigenetic modifying enzymes, recombinase, flippase, transposase, RNA-binding proteins (RBP), an Argonaute protein, any derivative thereof, any variant thereof, or any fragment thereof. In some embodiments, the actuator moiety comprises a Cas protein, and the system further comprises a guide RNA (gRNA) which complexes with the Cas protein. In some embodiments, the actuator moiety comprises an RBP complexed with a gRNA which is able to form a complex with a Cas protein. In some embodiments, the gRNA comprises a targeting segment which exhibits at least 80% sequence identity to a target polynucleotide. In some embodiments, the Cas protein substantially lacks DNA cleavage activity (i.e., dead Cas, deactivated Cas, or dCas). For example, the Cas protein is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity.

In some cases, a suitable actuator moiety comprises CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof, any variant thereof; and any fragment thereof.

A Cas protein referred to herein can be a type of protein or polypeptide. A Cas protein can refer to a nuclease. A Cas protein can refer to an endoribonuclease. A Cas protein can refer to any modified (e.g., shortened, mutated, lengthened) polypeptide sequence or homologue of the Cas protein. A Cas protein can be codon optimized. A Cas protein can be a codon-optimized homologue of a Cas protein. A Cas protein can be enzymatically inactive, partially active, constitutively active, fully active, inducible active and/or more active, (e.g. more than the wild type homologue of the protein or polypeptide). A Cas protein can be Cas9. A Cas protein can be Cpf1. A Cas protein can be C2c2. A Cas protein (e.g., variant, mutated, enzymatically inactive and/or conditionally enzymatically inactive site-directed polypeptide) can bind to a target nucleic acid. A Cas protein (e.g., variant, mutated, enzymatically inactive and/or conditionally enzymatically inactive endoribonuclease) can bind to a target RNA or DNA.

Non-limiting examples of Cas proteins include c2c1, C2c2, c2c3, Cas1, Cas1B, Cas2, Cas3, Cas4, Cas5, Cas5e (CasD), Cas6, Cashe, Cas6f, Cas7, Cas8a, Cas8a1, Cas8a2, Cas8b, Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

In some cases, a nuclease disclosed herein (e.g., Cas) can be a nucleic acid-guided nuclease (e.g., an RNA guided endonuclease). The term “guide nucleic acid” generally refers to a nucleic acid that can hybridize to another nucleic acid. A guide nucleic acid can be RNA. A guide nucleic acid can be DNA. The guide nucleic acid can be programmed to bind to a sequence of nucleic acid site-specifically. The nucleic acid to be targeted, or the target nucleic acid, can comprise nucleotides. The guide nucleic acid can comprise nucleotides. A portion of the target nucleic acid can be complementary to a portion of the guide nucleic acid. The strand of a double-stranded target polynucleotide that is complementary to and hybridizes with the guide nucleic acid can be called the complementary strand. The strand of the double-stranded target polynucleotide that is complementary to the complementary strand, and therefore may not be complementary to the guide nucleic acid can be called noncomplementary strand. A guide nucleic acid can comprise a polynucleotide chain and can be called a “single guide nucleic acid.” A guide nucleic acid can comprise two polynucleotide chains and can be called a “double guide nucleic acid.” If not otherwise specified, the term “guide nucleic acid” can be inclusive, referring to both single guide nucleic acids and double guide nucleic acids.

A guide nucleic acid can comprise a segment that can be referred to as a “nucleic acid-targeting segment” or a “nucleic acid-targeting sequence.” A nucleic acid-targeting segment can comprise a sub-segment that can be referred to as a “protein binding segment” or “protein binding sequence” or “Cas protein binding segment.”

A guide nucleic acid can comprise two separate nucleic acid molecules, which can be referred to as a double guide nucleic acid. A guide nucleic acid can comprise a single nucleic acid molecule, which can be referred to as a single guide nucleic acid (e.g., sgRNA). In some cases, the guide nucleic acid is a single guide nucleic acid comprising a fused CRISPR RNA (crRNA) and a transactivating crRNA (tracrRNA). In some cases, the guide nucleic acid is a single guide nucleic acid comprising a crRNA. In some cases, the guide nucleic acid is a single guide nucleic acid comprising a crRNA but lacking a tracrRNA. In some cases, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like and tracrRNA-like molecules.

The term “crRNA,” as used herein, generally refers to a nucleic acid with at least about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes). crRNA can generally refer to a nucleic acid with at most about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes). crRNA can refer to a modified form of a crRNA that can comprise a nucleotide change such as a deletion, insertion, or substitution, variant, mutation, or chimera. A crRNA can be a nucleic acid having at least about 60% sequence identity to a wild type exemplary crRNA (e.g., a crRNA from S. pyogenes) sequence over a stretch of at least 6 contiguous nucleotides. For example, a crRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary crRNA sequence (e.g., a crRNA from S. pyogenes) over a stretch of at least 6 contiguous nucleotides.

The term “tracrRNA,” as used herein, generally refers to a nucleic acid with at least about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes). tracrRNA can refer to a modified form of a tracrRNA that can comprise a nucleotide change such as a deletion, insertion, or substitution, variant, mutation, or chimera. A tracrRNA can refer to a nucleic acid that can be at least about 60% identical to a wild type exemplary tracrRNA (e.g., a tracrRNA from S. pyogenes) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes) sequence over a stretch of at least 6 contiguous nucleotides.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used.

In some cases, the effector domain can be a transcriptional activation domain selected from the group consisting of GAL4, VP16, VP64, p65, Rta, VPR, and variants thereof (e.g., mini-VPR). In some examples, the actuator moiety can be a Cas protein (e.g., dCas such as dCas9) fused to the transcriptional activation domain, as disclosed herein.

In some cases, the effector domain can be a transcriptional repressor domain selected from the group consisting of KRAB, SID, ERD, and variants thereof. In some examples, the actuator moiety can be a Cas protein (e.g., dCas such as dCas9) fused to the transcriptional repressor domain as disclosed herein.

In one aspect, the present disclosure provides a system comprising an actuator moiety, as disclosed herein, that is capable of binding a target polynucleotide sequence in a cell to regulate expression or activity of an endogenous cytokine (e.g., an interleukin (IL)) in the cell, as disclosed herein. In some cases, the actuator moiety is heterologous to the cell. For example, the IL can be IL-12 (e.g., IL-12A and/or IL-12B) or IL-21.

### V. Guide Nucleic Acid

In one aspect, the present disclosure provides a system comprising a guide nucleic acid molecule designed to bind a target polynucleotide sequence in a cell to regulate expression or activity of the target protein(s) in the cell, as disclosed herein. In some cases, the guide nucleic acid molecule can be capable of recruiting an actuator moiety to the target polynucleotide sequence in the cell, to regulate expression or activity of the target protein. In some cases, the system can comprise the actuator moiety. For example, the target protein(s) can comprise one or more proteins from Tables 1 and 2.

In some cases, the target polynucleotide sequence can be endogenous to the cell. In some cases, the TSS of a target gene encoding the target protein can be endogenous to the cell.

In some cases, the system can comprise at least or up to 2, at least or up to 3, at least or up to 3, at least or up to 4, at least or up to 5, at least or up to 6, at least or up to 7, at least or up to 8, at least or up to 9, or at least or up to 10 different guide nucleic acid molecules having different nucleic acid sequences. In some cases, the guide nucleic acid molecule can comprise a guide ribonucleic acid (RNA). In some examples, the system can comprise multi-plex guide nucleic acids (e.g., multi-plex guide RNAs).

In some cases, the system can comprise (i) a first guide nucleic acid molecule designed to bind a first target polynucleotide sequence of the target polynucleotide sequence as disclosed herein and (ii) a second guide nucleic acid molecule designed to bind a second target polynucleotide sequence of the target polynucleotide sequence as disclosed herein. In some examples, the system can comprise (i) a first guide nucleic acid molecule designed to bind a first portion of the TSS of the target gene encoding the target protein and (ii) a second guide nucleic acid molecule designed to bind a second portion of the TSS of the target gene encoding the target protein. In some examples, the first target polynucleotide sequence and the second target polynucleotide sequence can be separated by at least or up to about 1, at least or up to 2, at least or up to 3, at least or up to 3, at least or up to 4, at least or up to 5, at least or up to 6, at least or up to 7, at least or up to 8, at least or up to 9, at least or up to 10, at least or up to 15, at least or up to 20, at least or up to 30, at least or up to 40, at least or up to 50, at least or up to 60, at least or up to 70, at least or up to 80, at least or up to 90, at least or up to 100, at least or up to 200, at least or up to 300, at least or up to 400, at least or up to 500, at least or up to 600, at least or up to 700, at least or up to 800, at least or up to 900, at least or up to 1,000, at least or up to 2,000, at least or up to 3,000, at least or up to 4,000, or at least or up to 5,000 bases. The first target polynucleotide sequence and the second target polynucleotide sequence can be on a same strand of a target nucleic acid molecule (e.g., a target genome of the cell). Alternatively, the first target polynucleotide sequence and the second target polynucleotide sequence can be on different stands of the target nucleic acid molecule.

In some cases, the target gene encoding the target protein can comprise a plurality of TSSs comprising a first TSS and a second TSS. Each of the first TSS and the second TSS can encode different portions of the target gene. For example, the target protein can be a heterodimer, and the first TSS can be from a gene encoding a first monomer of the heterodimer, and the second TSS can be from a gene encoding a second monomer of the heterodimer. Thus, in some examples, the first guide nucleic acid molecule can (1a) comprise at least a portion of the first TSS or (1b) be at certain distance away from the first TSS as provided herein, and the second guide nucleic acid can (2a) comprise at least a portion of the second TSS or (2b) be at certain distance away from the second TSS as provided herein.

In some cases, the TSS (e.g., the first TSS) can have at least about 50%, at least about 60%, at least about 70%, at least about 80%, at least about 90%, at least about 95%, at least about 99%, or at least about 100% sequence identity to any one of the polynucleotide sequences provided in Table 3.

TABLE 3

| Gene | TSS +49 bp |
|---|---|
| ID3 | GTTGCAGGTCACTGTAGCGGGACTTCTTTTGGTTTTCTTTCTCTTT |
| | GGGG |
| c-Jun | ATTCCTGAAGCTGACAGCATTCGGGCCGAGATGTCTCGCTCCGT |
| | GGCCTT |
| TBX21/ | AGTGACAGCGGCCCGCTGGAGAGGAAGCCCGAGAGCTGCCGCG |
| T-bet | CGCCTGC |
| IL-21 | GCTGAAGTGAAAACGAGACCAAGGTCTAGCTCTACTGTTGGTAC |
| | TTATGA |
| TOX1 | CTCTTCTTCTTAAACAAACCACAAACGGATGTGAGGGAAGGAAG |
| | GTGTTT |
| TOX2 | ACTGCCCGCGGGAGCCGCCGCCGCCGCCGCCGCGCCCGCCATGG |
| | ACGTCC |
| SOCS-1 | AGAGCGAGCTGCGGCCGTGGCAGCTGCACGGCTCCTGGCCCCGG |
| | AGCATG |
| SHIP-1 | AGTTAAGCTGGTGGCAGCAGCCGAGGCCACCAAGAGGCAACGG |
| | GCGGCAG |
| BATF | AAAGCGAGCGACATGTCCCTTTGGGGAGCAGTCCCTCTGCACCC |
| | CAGAGT |
| B2M | ATTCCTGAAGCTGACAGCATTCGGGCCGAGATGTCTCGCTCCGT |
| | GGCCTT |
| TGFbR2 | ACTCGCGCGCACGGAGCGACGACACCCCCGCGCGTGCACCCGCT |
| | CGGGAC |

### VI. Delivery of Expression of the System in a Cell

In one aspect, the present disclosure provides a cell (e.g., an immune cell) comprising (or expressing) any of the subject system disclosed herein.

In one aspect, the present disclosure provides a population of cells (e.g., a population of immune cells) comprising (or expressing) any of the subject system disclosed herein.

RNA or DNA viral based systems can be used to deliver one or more genes that encode the any of the polypeptides and/or polynucleotides disclosed herein (e.g., chimeric receptor, chimeric adaptor, actuator moiety with or without the effector domain, or a gene encoding thereof) to the cell of the present disclosure. Viral vectors can be used to treat cells in vitro, and the modified cells can optionally be administered (ex vivo). Alternatively, viral vectors can be administered directly (in vivo) to the subject. Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted transgene.

In some cases, non-viral delivery methods can be used to deliver any of the polypeptides and/or polynucleotides disclosed herein (e.g., chimeric receptor, chimeric adaptor, actuator moiety with or without the effector domain, or a gene encoding thereof) to the cell of the present disclosure. Methods of non-viral delivery of such cargo can include lipofection, nucleofection, microinjection, biolistics, virosomes, liposomes, immunoliposomes, exosomes, polycation or lipid: cargo conjugates (or aggregates), naked polypeptide (e.g., recombinant polypeptides), naked DNA, artificial virions, and agent-enhanced uptake of polypeptide or DNA. Cationic and neutral lipids that are suitable for efficient receptor-recognition lipo-delivery of polynucleotides or polypeptides can be used.

### VII. Methods and Compositions

In one aspect, the present disclosure provides a method of conditionally regulating expression or activity of a target protein (e.g., endogenous target protein(s) 33) of a cell by introducing (or expressing) any of the subject system as disclosed herein.

In one aspect, the present disclosure provides a method of conditionally regulating expression or activity of a target protein (e.g., endogenous target protein(s)) of a cell. The method can comprise (a) exposing a chimeric receptor polypeptide (receptor) to a ligand, wherein the receptor undergoes a modification upon binding to the ligand. The method can comprise (b) in response to the receptor modification, forming a complex between an actuator moiety and a target polynucleotide sequence, as disclosed herein, to regulate expression or activity of the target protein.

In some cases, the method further comprises administering a co-therapeutic agent.

In some cases, the cell administered to the subject can be autologous or allogeneic to the subject. For example, the cell administered to the subject can be an autologous immune cell or an allogeneic immune cell.

In one aspect, the present disclosure provides a composition comprising the cell or the population of cells (e.g., population of engineered immune cells) that comprises (or expresses) any of the subject system as disclosed herein. The composition can be administered to the subject to treat a condition (e.g., cancer, tumor) of the subject. The composition can comprise at least or up to about 1 dose, at least or up to about 2 doses, at least or up to about 3 doses, at least or up to about 4 doses, at least or up to about 5 doses, at least or up to about 6 doses, at least or up to about 7 doses, at least or up to about 8 doses, at least or up to about 9 doses, or at least or up to about 10 doses.

In some cases, the composition further comprises a co-therapeutic agent.

The composition as disclosed herein can be a pharmaceutical composition. The pharmaceutical composition can be in any suitable form, (depending upon the desired method of administration). The pharmaceutical composition cam be provided in unit dosage form, can be provided in a sealed container, and/or can be provided as part of a kit. Such a kit can include instructions for use. The kit can include a plurality of said unit dosage forms.

Non-limiting examples of a co-therapeutic agent can include cytotoxic agents, chemotherapeutic agents, growth inhibitory agents, agents used in radiation therapy, anti-angiogenesis agents, apoptotic agents, anti-tubulin agents, and other agents to treat cancer, for example, anti-CD20 antibodies, anti-PD1 antibodies (e.g., Pembrolizumab) platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets PDGFR-β, BlyS, APRIL, BCMA receptor(s), TRAIL/Apo2, other bioactive and organic chemical agents, and the like.

The term “cytotoxic agent” generally refers to a substance that inhibits or prevents the function of cells and/or causes destruction of cells. Non-limiting examples of a cytotoxic agent can include radioactive isotopes (e.g., At211, 1131, 1125, Y90, Re186, Re188, Sm153, Bi212, P32, and radioactive isotopes of Lu), chemotherapeutic agents, e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin.

Non-limiting examples of a chemotherapeutic agent can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics; dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aidophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, for example taxanes including TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhône-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin. Additional chemotherapeutic agents include the cytotoxic agents useful as antibody drug conjugates, such as maytansinoids (DM1, for example) and the auristatins MMAE and MMAF, for example.

Examples of a chemotherapeutic agent can also include “anti-hormonal agents” or “endocrine therapeutics” that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD) leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4 (5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGFR); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

Examples of a chemotherapeutic agent can also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®), Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, feMzumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1λ antibody genetically modified to recognize interleukin-12 p40 protein.

Examples of a chemotherapeutic agent can also include “tyrosine kinase inhibitors” such as an EGFR-targeting agent (e.g., small molecule, antibody, etc.); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino) phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®).

Examples of a chemotherapeutic agent can also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Examples of a chemotherapeutic agent can also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluo-cinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate: immune selective anti-inflammatory peptides (ImSAIDs) such as phenylalanine-glutamine-glycine (FEG) and its D-isomeric form (feG) (IMULAN BioTher-apeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (ENBREL®), infliximab (REMICADE®), adalimumab (HUMIRA®), certolizumab pegol (CIMZIA®), golimumab (SIMPONI®), Interleukin 1 (IL-1) blockers such as anakinra (KINERET®), T-cell costimulation blockers such as abatacept (ORENCIA®), Interleukin 6 (IL-6) blockers such as tocilizumab (ACTEMERA®); Interleukin 13 (IL-13) blockers such as lebrikizumab; Interferon alpha (IFN) blockers such as rontalizumab; beta 7 integrin blockers such as rhuMAb Beta7; IgE pathway blockers such as Anti-M1 prime; Secreted homotrimeric LTa3 and membrane bound heterotrimer LTa/β2 blockers such as Anti-lymphotoxin alpha (LTa); miscellaneous investigational agents such as thioplatin, PS-341, phenylbutyrate, ET-18-OCH₃, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifamib (R11577); orafenib, ABT510; Bcl-2 inhibitor such as oblimersen sodium (GENASENSE®); pixantrone; famesyltransferase inhibitors such as lonafamib (SCH 6636, SARASAR™); and pharmaceutically acceptable salts, acids or derivatives of any of the above; as well as combinations of two or more of the above.

The term “growth inhibitory agent” generally refers to a compound or composition which inhibits growth and/or proliferation of a cell (e.g., a cell whose growth is dependent on PD-L1 expression) either in vitro or in vivo. The growth inhibitory agent may be one which significantly reduces the percentage of cells in S phase. Non-limiting examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as the anthracycline antibiotic doxorubicin ((8S-cis)-10-[(3-amino-2,3,6-trideoxy-α-L-lyxo-hexapyranosyl)oxy]-7,8,9,10-tetrahydr 0-6,8,11-trihydroxy-8-(hydroxyacetyl)-1-methoxy-5,12-naphthacenedione), epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

### VIII. Therapeutic Applications

A subject system can be introduced in a variety of immune cells, including any cell that is involved in an immune response. In some embodiments, immune cells comprise granulocytes such as asophils, eosinophils, and neutrophils; mast cells; monocytes which can develop into macrophages; antigen-presenting cells such as dendritic cells; and lymphocytes such as natural killer cells (NK cells), B cells, and T cells. In some embodiments, an immune cell is an immune effector cell. An immune effector cell refers to an immune cell that can perform a specific function in response to a stimulus. In some embodiments, an immune cell is an immune effector cell which can induce cell death. In some embodiments, the immune cell is a lymphocyte. In some embodiments, the lymphocyte is a NK cell. In some embodiments the lymphocyte is a T cell. In some embodiments, the T cell is an activated T cell. T cells include both naive and memory cells (e.g. central memory or T_{CM}, effector memory or T_{EM} and effector memory RA or T_{EMRA}), effector cells (e.g. cytotoxic T cells or CTLs or Tc cells), helper cells (e.g. Th1, Th2, Th3, Th9, Th7, TFH), regulatory cells (e.g. Treg, and Tr1 cells), natural killer T cells (NKT cells), tumor infiltrating lymphocytes (TILs), lymphocyte-activated killer cells (LAKs), αβ T cells, γδ T cells, and similar unique classes of the T cell lineage. T cells can be divided into two broad categories: CD8+ T cells and CD4+ T cells, based on which protein is present on the cell's surface. T cells expressing a subject system can carry out multiple functions, including killing infected cells and activating or recruiting other immune cells. CD8+ T cells are referred to as cytotoxic T cells or cytotoxic T lymphocytes (CTLs). CTLs expressing a subject system can be involved in recognizing and removing virus-infected cells and cancer cells. CTLs have specialized compartments, or granules, containing cytotoxins that cause apoptosis, e.g., programmed cell death. CD4+ T cells can be subdivided into four sub-sets-Th1, Th2, Th17, and Treg, with “Th” referring to “T helper cell,” although additional sub-sets may exist. Th1 cells can coordinate immune responses against intracellular microbes, especially bacteria. They can produce and secrete molecules that alert and activate other immune cells, like bacteria-ingesting macrophages. Th2 cells are involved in coordinating immune responses against extracellular pathogens, like helminths (parasitic worms), by alerting B cells, granulocytes, and mast cells. Th17 cells can produce interleukin 17 (IL-17), a signaling molecule that activates immune and non-immune cells. Th17 cells are important for recruiting neutrophils.

A ligand or an antigen (i.e., a target antigen) of an antigen binding moiety as disclosed herein can be a cell surface marker, a secreted marker, or an intracellular marker.

Non-limiting examples of an antigen (i.e., a target antigen) of an antigen binding moiety as disclosed herein can include ADGRE2, carbonic anhydrase IX (CA1X), CCRI, CCR4, carcinoembryonic antigen (CEA), CD3ζ, CD5, CD7, CD8, CD10, CD19, CD20, CD22, CD30, CD33, CD34, CD38, CD41, CD44, CD44V6, CD49f, CD56, CD70, CD74, CD99, CD123, CD133, CD138, CD269 (BCMA), CD S, CLEC12A, an antigen of a cytomegalovirus (CMV) infected cell (e.g., a cell surface antigen), epithelial glycoprotein2 (EGP 2), epithelial glycoprotein-40 (EGP-40), ep-ithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-a, Ganglioside G2 (GD2), Ganglioside G3 (GD3), gp100, human Epidermal Growth Factor Receptor 2 (HER-2), human telomerase reverse transcriptase (hTERT), ICAM-1, Integrin B7, Interleukin-13 receptor subunit alpha-2 (IL-13Rα2), κ-light chain, kinase insert domain receptor (KDR), Kappa, Lewis A (CA19.9), Lewis Y (LeY), L1 cell adhesion molecule (L1-CAM), LILRB2, MART-1, melanoma antigen family A 1 (MAGE-A1), MICA/B, Mucin 1 (Muc-1), Mucin 16 (Muc-16), Mesothelin (MSLN), NKCSI, NKG2D ligand, c-Met, cancer-testis antigen NY-ESO-1, NY-ESO-2, oncofetal antigen (h5T4), PRAIVIE, prostate stem cell antigen (PSCA), PRAME prostate-specific membrane antigen (PSMA), ROR1, tumor-associated glycoprotein 72 (TAG-72), TIM-3, TRBCI, TRBC2, vascular endothelial growth factor R2 (VEGF-R2), Wilms tumor protein (WT-1), and various pathogen antigen (e.g., pathogen antigens derived from a virus, bacteria, fungi, parasite and protozoa capable of causing diseases). In some examples, a pathogen antigen is derived from HIV, HBV, EBV, HPV, Lasse Virus, Influenza Virus, or Coronavirus.

Additional examples of the antigen of the antigen binding moiety as disclosed herein can include 1-40-β-amyloid, 4-1BB, 5AC, 5T4, activin receptor-like kinase 1, ACVR2B, adenocarcinoma antigen, AGS-22M6, alpha-fetoprotein, angiopoietin 2, angiopoietin 3, anthrax toxin, AOC3 (VAP-1), B7-H3, Bacillus anthracis anthrax, BAFF, beta-amyloid, B-lymphoma cell, C242 antigen, C5, CA-125, Canis lupus familiaris IL31, carbonic anhydrase 9 (CA-IX), cardiac myosin, CCL11 (eotaxin-1), CCR4, CCR5, CD11, CD18, CD125, CD140a, CD147 (basigin), CD15, CD152, CD154 (CD40L), CD19, CD2, CD20, CD200, CD22, CD221, CD23 (IgE receptor), CD25 (a chain of IL-2 receptor), CD27, CD274, CD28, CD3, CD3 epsilon, CD30, CD33, CD37, CD38, CD4, CD40, CD40 ligand, CD41, CD44 v6, CD5, CD51, CD52, CD56, CD6, CD70, CD74, CD79B, CD80, CEA, CEA-related antigen, CFD, ch4D5, CLDN18.2, Clostridium difficile, clumping factor A, CSFIR, CSF2, CTLA-4, C-X-C chemokine receptor type 4, cytomegalovirus, cytomegalovirus glycoprotein B, dabigatran, DLL4, DPP4, DR5, E. coli shiga toxin type-1, E. coli shiga toxin type-2, EGFL7, EGFR, endotoxin, EpCAM, episialin, ERBB3, Escherichia coli, F protein of respiratory syncytial virus, FAP, fibrin II beta chain, fibronectin extra domain-B, folate hydrolase, folate receptor 1, folate receptor alpha, Frizzled receptor, ganglioside GD2, GD2, GD3 ganglioside, glypican 3, GMCSF receptor α-chain, GPNMB, growth differentiation factor 8, GUCY2C, hemagglutinin, hepatitis B surface antigen, hepatitis B virus, HER1, HER2/neu, HER3, HGF, HHGFR, histone complex, HIV-1, HLA-DR, HNGF, Hsp90, human scatter factor receptor kinase, human TNF, human beta-amyloid, ICAM-1 (CD54), IFN-α, IFN-γ, IgE, IgE Fc region, IGF-1 receptor, IGF-1, IGHE, IL17A, IL17F, IL20, IL-12, IL-13, IL-17, IL-1B, IL-22, IL-23, IL-31RA, IL-4, IL-5, IL-6, IL-6 receptor, IL-9, ILGF2, influenza A hemagglutinin, influenza A virus hemagglutinin, insulin-like growth factor I receptor, integrin α4β7, integrin α4, integrin α5β1, integrin α7β7, integrin αIIbβ3, integrin αvβ3, interferon α/β receptor, interferon gamma-induced protein, ITGA2, ITGB2 (CD18), KIR2D, Lewis-Y antigen, LFA-1 (CD11a), LINGO-1, lipoteichoic acid, LOXL2, L-selectin (CD62L), LTA, MCP-1, mesothelin, MIF, MS4A1, MSLN, MUC1, mucin CanAg, myelin-associated glycoprotein, myostatin, NCA-90 (granulocyte antigen), neural apoptosis-regulated proteinase 1, NGF, N-glycolylneuraminic acid, NOGO-A, Notch receptor, NRP1, Oryctolagus cuniculus, OX-40, oxLDL, PCSK9, PD-1, PDCD1, PDGF-R a, phosphate-sodium co-transporter, phosphatidylserine, platelet-derived growth factor receptor beta, prostatic carcinoma cells, Pseudomonas aeruginosa, rabies virus glycoprotein, RANKL, respiratory syncytial virus, RHD, Rhesus factor, RON, RTN4, sclerostin, SDC1, selectin P, SLAMF7, SOST, sphingosine-1-phosphate, Staphylococcus aureus, STEAP1, TAG-72, T-cell receptor, TEM1, tenascin C, TFPI, TGF-β 1, TGF-2, TGF-β, TNF-α, TRAIL-R1, TRAIL-R2, tumor antigen CTAA16.88, tumor specific glycosylation of MUC1, tumor-associated calcium signal transducer 2, TWEAK receptor, TYRP1 (glycoprotein 75), VEGFA, VEGFR1, VEGFR2, vimentin, and VWF.

Additional examples of the antigen of the antigen binding moiety as disclosed herein can include 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (ela2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRVIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and k-light chain.

Additional examples of the antigen of the antigen binding moiety as disclosed herein can include an antibody, a fragment thereof, or a variant thereof. Such antibody can be a natural antibody (e.g., naturally secreted by a subject's immune cell, such as B cells), a synthetic antibody, or a modified antibody. In some cases, the antigen of the antigen binding moiety as disclosed herein can include an Fc domain of an antibody from the group comprising 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, zlintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatumumab, concizumab, Cotara (iodine I-131 derlotuximab biotin), cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glem-batumumab vedotin, GNR-006, GNR-011, golimumab, gomiliximab, GSK2849330, GSK2857916, GSK3174998, GSK3359609, guselkumab, Hu14.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imciromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L19IL2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDI0680, MEDI-3617, MEDI-551 (inebilizumab), MEDI-565, MEDI6469, mepolizumab, metelimumab, MGB453, MGD006/S80880, MGD007, MGD009, MGD011, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, RO7009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIVIA, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRBS07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, and zolimomab aritox.

Any of the systems disclosed herein can be utilized to regulate expression or activity of an endogenous protein of a cell. Example genes encoding the endogenous protein as disclosed herein are provided in Tables 4, 5, and 6. Exemplary genes associated with certain diseases and disorders are provided in Tables 4 and 5. Examples of signaling biochemical pathway-associated genes and polynucleotides are listed in Table 6.

TABLE 4

| DISEASE/DISORDERS | GENE(S) |
|---|---|
| Neoplasia | PTEN; ATM; ATR; EGFR; ERBB2; ERBB3; ERBB4; |
| | Notch1; Notch2; Notch3; Notch4; AKT; AKT2; AKT3; |
| | HIF; |
| | HIF1a; HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 |
| | (4 |
| | variants); Igf2 (3 variants); Igf 1 Receptor; Igf 2 Receptor; |
| | Bax; Bcl2; caspases family (9 members: |
| | 1, 2, 3, 4, 6, 7, 8, 9, 12); Kras; Apc |
| Age-related Macular | Abcr; Cc12; Cc2; cp (ceruloplasmin); Timp3; cathepsinD; |
| Degeneration | Vldlr; Ccr2 |
| Schizophrenia | Neuregulin1 (Nrg1); Erb4 (receptor for Neuregulin); |
| | Complexin1 (Cplx1); Tph1 Tryptophan hydroxylase; |
| | Tph2 |
| | Tryptophan hydroxylase 2; Neurexin 1; GSK3; GSK3a; |
| | GSK3b |
| Disorders | 5-HTT (Slc6a4); COMT; DRD (Drd1a); SLC6A3; |
| | DAOA; |
| | DTNBP1; Dao (Dao1) |
| Trinucleotide Repeat | HTT (Huntington's Dx); SBMA/SMAX1/AR (Kennedy's |
| Disorders | Dx); FXN/X25 (Friedrich's Ataxia); ATX3 (Machado- |
| | Joseph's Dx); ATXN1 and ATXN2 (spinocerebellar |
| | ataxias); DMPK (myotonic dystrophy); Atrophin-1 and |
| | Atn1 |
| | (DRPLA Dx); CBP (Creb-BP - global instability); |
| | VLDLR |
| | (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin |
| Disorders | (Ncstn); PEN-2 |
| Others | Nos1; Parp1; Nat1; Nat2 |
| Prion - related disorders | Prp |
| ALS | SOD1; ALS2; STEX; FUS; TARDBP; VEGF (VEGF-a; |
| | VEGF-b; VEGF-c) |
| Drug addiction | Prkce (alcohol); Drd2; Drd4; ABAT (alcohol); GRIA2; |
| | Grm5; Grin1; Htr1b; Grin2a; Drd3; Pdyn; Gria1 (alcohol) |
| Autism | Mecp2; BZRAP1; MDGA2; Sema5A; Neurexin 1; Fragile |
| | X |
| | (FMR2 (AFF2); FXR1; FXR2; Mglur5) |
| Alzheimer's Disease | E1; CHIP; UCH; UBB; Tau; LRP; PICALM; Clusterin; |
| | PS1; |
| | SORL1; CR1; Vldlr; Uba1; Uba3; CHIP28 (Aqp1, |
| | Aquaporin 1); Uchl1; Uch13; APP |
| Inflammation | IL-10; IL-1 (IL-la; IL-1b); IL-13; IL-17 (IL-17a (CTLA8); |
| | IL- |
| | 17b; IL-17c; IL-17d; IL-17f); II-23; Cx3cr1; ptpn22; |
| | TNFa; |
| | NOD2/CARD15 for IBD; IL-6; IL-12 (IL-12a; IL-12b); |
| | CTLA4; Cx3cl1 |
| Parkinson's Disease | x-Synuclein; DJ-1; LRRK2; Parkin; PINK1 |

TABLE 5

| | |
|---|---|
| Blood and | Anemia (CDAN1, CDA1, RPS19, DBA, PKLR, PK1, NT5C3, |
| coagulation diseases | UMPH1, |
| and disorders | PSN1, RHAG, RH50A, NRAMP2, SPTB, ALAS2, ANH1, ASB, |
| | ABCB7, ABC7, ASAT); Bare lymphocyte syndrome (TAPBP, |
| | TPSN, |
| | TAP2, ABCB3, PSF2, RING11, MHC2TA, C2TA, RFX5, |
| | RFXAP, |
| | RFX5), Bleeding disorders (TBXA2R, P2RX1, P2X1); Factor H |
| | and |
| | factor H-like 1 (HF1, CFH, HUS); Factor V and factor VIII |
| | (MCFD2); |
| | Factor VII deficiency (F7); Factor X deficiency (F10); Factor XI |
| | deficiency (F11); Factor XII deficiency (F12, HAF); Factor |
| | XIIIA |
| | deficiency (F13A1, F13A); Factor XIIIB deficiency (F13B); |
| | Fanconi |
| | anemia (FANCA, FACA, FA1, FA, FAA, FAAP95, FAAP90, |
| | FLJ34064, |
| | FANCB, FANCC, FACC, BRCA2, FANCD1, FANCD2, |
| | FANCD, |
| | FACD, FAD, FANCE, FACE, FANCF, XRCC9, FANCG, |
| | BRIP1, |
| | BACH1, FANCJ, PHF9, FANCL, FANCM, KIAA1596); |
| | Hemophagocytic lymphohistiocytosis disorders (PRF1, HPLH2, |
| | UNC13D, MUNC13-4, HPLH3, HLH3, FHL3); Hemophilia A |
| | (F8, F8C, |
| | HEMA); Hemophilia B (F9, HEMB), Hemorrhagic disorders (PI, |
| | ATT, |
| | F5); Leukocyde deficiencies and disorders (ITGB2, CD18, |
| | LCAMB, |
| | LAD, EIF2B1, EIF2BA, EIF2B2, EIF2B3, EIF2B5, LVWM, |
| | CACH, |
| | CLE, EIF2B4); Sickle cell anemia (HBB); Thalassemia (HBA2, |
| | HBB, HBD, LCRB, HBA1). |
| Cell dysregulation | B-cell non-Hodgkin lymphoma (BCL7A, BCL7); Leukemia |
| and oncology | (TAL1 |
| diseases and | TCL5, SCL, TAL2, FLT3, NBS1, NBS, ZNFN1A1, IK1, LYF1, |
| disorders | HOXD4, HOX4B, BCR, CML, PHL, ALL, ARNT, KRAS2, |
| | RASK2, |
| | GMPS, AF10, ARHGEF12, LARG, KIAA0382, CALM, CLTH, |
| | CEBPA, CEBP, CHIC2, BTL, FLT3, KIT, PBT, LPP, NPM1, |
| | NUP214, |
| | D9S46E, CAN, CAIN, RUNX1, CBFA2, AML1, WHSCIL1, |
| | NSD3, |
| | FLT3, AF1Q, NPM1, NUMA1, ZNF145, PLZF, PML, MYL, |
| | STAT5B, |
| | AF10, CALM, CLTH, ARL11, ARLTS1, P2RX7, P2X7, BCR, |
| | CML, |
| | PHL, ALL, GRAF, NF1, VRNF, WSS, NFNS, PTPN11, PTP2C, |
| | SHP2, |
| | NS1, BCL2, CCND1, PRAD1, BCL1, TCRA, GATA1, GF1, |
| | ERYF1, |
| | NFE1, ABL1, NQO1, DIA4, NMOR1, NUP214, D9S46E, CAN, |
| | CAIN). |
| Inflammation and | AIDS (KIR3DL1, NKAT3, NKB1, AMB11, KIR3DS1, IFNG, |
| immune related | CXCL12, |
| diseases and | SDF1); Autoimmune lymphoproliferative syndrome (TNFRSF6, |
| disorders | APT1, |
| | FAS, CD95, ALPS1A); Combined immunodeficiency, (IL2RG, |
| | SCIDX1, SCIDX, IMD4); HIV-1 (CCL5, SCYA5, D17S136E, |
| | TCP228), |
| | HIV susceptibility or infection (IL10, CSIF, CMKBR2, CCR2, |
| | CMKBR5, CCCKR5 (CCR5)); Immunodeficiencies (CD3E, |
| | CD3G, |
| | AICDA, AID, HIGM2, TNFRSF5, CD40, UNG, DGU, HIGM4, |
| | TNFSF5, CD40LG, HIGM1, IGM, FOXP3, IPEX, AIID, XPID, |
| | PIDX, |
| | TNFRSF14B, TACI); Inflammation (IL-10, IL-1 (IL-1a, IL-1b), |
| | IL-13, |
| | IL-17 (IL-17a (CTLA8), IL-17b, IL-17c, IL-17d, IL-17f), II-23, |
| | Cx3cr1, |
| | ptpn22, TNFa, NOD2/CARD15 for IBD, IL-6, IL-12 (IL-12a, IL- |
| | 12b), |
| | CTLA4, Cx3cl1); Severe combined immunodeficiencies |
| | (SCIDs)(JAK3, |
| | JAKL, DCLRE1C, ARTEMIS, SCIDA, RAG1, RAG2, ADA, |
| | PTPRC, |
| | CD45, LCA, IL7R, CD3D, T3D, IL2RG, SCIDX1, SCIDX, |
| | IMD4). |
| Metabolic, liver, | Amyloid neuropathy (TTR, PALB); Amyloidosis (APOA1, APP, |
| kidney and protein | AAA, |
| diseases and | CVAP, AD1, GSN, FGA, LYZ, TTR, PALB); Cirrhosis (KRT18, |
| disorders | KRT8, |
| | CIRH1A, NAIC, TEX292, KIAA1988); Cystic fibrosis (CFTR, |
| | ABCC7, |
| | CF, MRP7); Glycogen storage diseases (SLC2A2, GLUT2, |
| | G6PC, |
| | G6PT, G6PT1, GAA, LAMP2, LAMPB, AGL, GDE, GBE1, |
| | GYS2, |
| | PYGL, PFKM); Hepatic adenoma, 142330 (TCF1, HNF1A, |
| | MODY3), |
| | Hepatic failure, early onset, and neurologic disorder (SCOD1, |
| | SCO1), |
| | Hepatic lipase deficiency (LIPC), Hepatoblastoma, cancer and |
| | carcinomas (CTNNB1, PDGFRL, PDGRL, PRLTS, AXIN1, |
| | AXIN, |
| | CTNNB1, TP53, P53, LFS1, IGF2R, MPRI, MET, CASP8, |
| | MCH5; |
| | Medullary cystic kidney disease (UMOD, HNFJ, FJHN, |
| | MCKD2, |
| | ADMCKD2); Phenylketonuria (PAH, PKU1, QDPR, DHPR, |
| | PTS); |
| | Polycystic kidney and hepatic disease (FCYT, PKHD1, ARPKD, |
| | PKD1, |
| | PKD2, PKD4, PKDTS, PRKCSH, G19P1, PCLD, SEC63). |
| Muscular/Skeletal | Becker muscular dystrophy (DMD, BMD, MYF6), Duchenne |
| diseases and | Muscular |
| disorders | Dystrophy (DMD, BMD); Emery-Dreifuss muscular dystrophy |
| | (LMNA, |
| | LMN1, EMD2, FPLD, CMD1A, HGPS, LGMD1B, LMNA, |
| | LMN1, |
| | EMD2, FPLD, CMD1A); Facioscapulohumeral muscular |
| | dystrophy |
| | (FSHMD1A, FSHD1A); Muscular dystrophy (FKRP, MDCIC, |
| | LGMD2I, LAMA2, LAMM, LARGE, KIAA0609, MDC1D, |
| | FCMD, |
| | TTID, MYOT, CAPN3, CANP3, DYSF, LGMD2B, SGCG, |
| | LGMD2C, |
| | DMDA1, SCG3, SGCA, ADL, DAG2, LGMD2D, DMDA2, |
| | SGCB, |
| | LGMD2E, SGCD, SGD, LGMD2F, CMD1L, TCAP, LGMD2G, |
| | CMDIN, TRIM32, HT2A, LGMD2H, FKRP, MDC1C, LGMD2I, |
| | TTN, |
| | CMD1G, TMD, LGMD2J, POMT1, CAV3, LGMD1C, SEPN1, |
| | SELN, |
| | RSMD1, PLEC1, PLTN, EBS1); Osteopetrosis (LRP5, BMND1, |
| | LRP7, |
| | LR3, OPPG, VBCH2, CLCN7, CLC7, OPTA2, OSTM1, GL, |
| | TCIRG1, |
| | TIRC7, OC116, OPTB1); Muscular atrophy (VAPB, VAPC, |
| | ALS8, |
| | SMN1, SMA1, SMA2, SMA3, SMA4, BSCL2, SPG17, GARS, |
| | SMAD1, |
| | CMT2D, HEXB, IGHMBP2, SMUBP2, CATF1, SMARD1). |
| Neurological and | ALS (SOD1, ALS2, STEX, FUS, TARDBP, VEGF (VEGF-a, |
| neuronal diseases | VEGF-b, |
| and disorders | VEGF-c); Alzheimer disease (APP, AAA, CVAP, AD1, APOE, |
| | AD2, |
| | PSEN2, AD4, STM2, APBB2, FE65L1, NOS3, PLAU, URK, |
| | ACE, |
| | DCP1, ACE1, MPO, PACIP1, PAXIP1L, PTIP, A2M, BLMH, |
| | BMH, |
| | PSEN1, AD3); Autism (Mecp2, BZRAP1, MDGA2, Sema5A, |
| | Neurexin |
| | 1, GLO1, MECP2, RTT, PPMX, MRX16, MRX79, NLGN3, |
| | NLGN4, |
| | KIAA1260, AUTSX2); Fragile X Syndrome (FMR2, FXR1, |
| | FXR2, |
| | mGLUR5); Huntington's disease and disease like disorders (HD, |
| | IT15, |
| | PRNP, PRIP, JPH3, JP3, HDL2, TBP, SCA17); Parkinson |
| | disease |
| | (NR4A2, NURR1, NOT, TINUR, SNCAIP, TBP, SCA17, |
| | SNCA, |
| | NACP, PARK1, PARK4, DJ1, PARK7, LRRK2, PARK8, |
| | PINK1, |
| | PARK6, UCHL1, PARK5, SNCA, NACP, PARK1, PARK4, |
| | PRKN, |
| | PARK2, PDJ, DBH, NDUFV2); Rett syndrome (MECP2, RTT, |
| | PPMX, |
| | MRX16, MRX79, CDKL5, STK9, MECP2, RTT, PPMX, |
| | MRX16, |
| | MRX79, x-Synuclein, DJ-1); Schizophrenia (Neuregulin1 (Nrg1), |
| | Erb4 |
| | (receptor for Neuregulin), Complexin1 (Cplx1), Tph1 Tryptophan |
| | hydroxylase, Tph2, Tryptophan hydroxylase 2, Neurexin 1, |
| | GSK3, |
| | GSK3a, GSK3b, 5-HTT (Slc6a4), COMT, DRD (Drd1a), |
| | SLC6A3, |
| | DAOA, DTNBP1, Dao (Dao1)); Secretase Related Disorders |
| | (APH-1 |
| | (alpha and beta), Presenilin (Psen1), nicastrin, (Ncstn), PEN-2, |
| | Nos1, |
| | Parp1, Nat1, Nat2); Trinucleotide Repeat Disorders (HTT |
| | (Huntington's |
| | Dx), SBMA/SMAX1/AR (Kennedy's Dx), FXN/X25 (Friedrich's |
| | Ataxia), ATX3 (Machado- Joseph's Dx), ATXN1 and ATXN2 |
| | (spinocerebellar ataxias), DMPK (myotonic dystrophy), Atrophin- |
| | 1 and |
| | Atn1 (DRPLA Dx), CBP (Creb-BP - global instability), VLDLR |
| | (Alzheimer's), Atxn7, Atxn10). |
| Ocular diseases | Age-related macular degeneration (Aber, Cc12, Cc2, cp |
| and disorders | (ceruloplasmin), |
| | Timp3, cathepsinD, Vldlr, Ccr2); Cataract (CRYAA, CRYA1, |
| | CRYBB2, |
| | CRYB2, PITX3, BFSP2, CP49, CP47, CRYAA, CRYA1, PAX6, |
| | AN2, |
| | MGDA, CRYBA1, CRYB1, CRYGC, CRYG3, CCL, LIM2, |
| | MP19, |
| | CRYGD, CRYG4, BFSP2, CP49, CP47, HSF4, CTM, HSF4, |
| | CTM, |
| | MIP, AQPO, CRYAB, CRYA2, CTPP2, CRYBB1, CRYGD, |
| | CRYG4, |
| | CRYBB2, CRYB2, CRYGC, CRYG3, CCL, CRYAA, CRYA1, |
| | GJA8, |
| | CX50, CAE1, GJA3, CX46, CZP3, CAE3, CCM1, CAM, |
| | KRIT1); |
| | Corneal clouding and dystrophy (APOA1, TGFBI, CSD2, |
| | CDGG1, |
| | CSD, BIGH3, CDG2, TACSTD2, TROP2, M1S1, VSX1, RINX, |
| | PPCD, |
| | PPD, KTCN, COL8A2, FECD, PPCD2, PIP5K3, CFD); Cornea |
| | plana |
| | congenital (KERA, CNA2); Glaucoma (MYOC, TIGR, GLC1A, |
| | JOAG, |
| | GPOA, OPTN, GLCIE, FIP2, HYPL, NRP, CYP1B1, GLC3A, |
| | OPA1, |
| | NTG, NPG, CYP1B1, GLC3A); Leber congenital amaurosis |
| | (CRB1, |
| | RP12, CRX, CORD2, CRD, RPGRIP1, LCA6, CORD9, RPE65, |
| | RP20, |
| | AIPL1, LCA4, GUCY2D, GUC2D, LCA1, CORD6, RDH12, |
| | LCA3); |
| | Macular dystrophy (ELOVL4, ADMD, STGD2, STGD3, RDS, |
| | RP7, PRPH2, PRPH, AVMD, AOFMD, VMD2). |

TABLE 6

| CELLULAR FUNCTION | GENES |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; E1F4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| | PRKAA2; EIF2AK2; RAC1; RAP1A; GRK6; ROCK2; |
| | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4, AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton Signaling | ACTN4; PRKCE; ITGAM; ROCK1; ITGA5; IRAK1; |
| | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease Signaling | PRKCE; IGF1; EP300; RCOR1; PRKCZ; HDAC4; TGM2; |
| | MAPK1; CAPNS1; AKT2; EGFR; NCOR2; SP1; CAPN2; |
| | PIK3CA; HDAC5; CREB1; PRKC1; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Aryl Hydrocarbon Receptor | HSPB1; EP300; FASN; TGM2; RXRA; MAPK1; NQO1; |
| Signaling | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ: TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4: PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5: PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| Wnt & Beta catenin Signaling | CD44; EP300; LRP6; DVL3; CSNK1E; GJA1; SMO; |
| | AKT2; PIN1; CDH1; BTRC; GNAQ; MARK2; PPP2R1A; |
| | WNT11; SRC; DKK1; PPP2CA; SOX6; SFRP2: ILK; |
| | LEF1; SOX9; TP53; MAP3K7; CREBBP; TCF (e.g., TCF7, |
| | TCF7L2); AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2: MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-Mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled Receptor | PRKCE; RAP1A; RGS16; MAPK1; GNAS; AKT2; IKBKB; |
| Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate Metabolism | PRKCE; IRAK1; PRKAA2; EIF2AK2; PTEN; GRK6; |
| | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S Checkpoint | HDAC4; SMAD3; SUV39H1; HDAC5; CDKN1B; BTRC; |
| Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA, PIK3C2A; BTK; LCK; RAF1; IKBKG; RELB, FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral Sclerosis | BID; IGF1; RAC1; BIRC4; PGF; CAPNS1; CAPN2; |
| Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor Signaling | TAF4B; EP300; CARM1; PCAF; MAPK1; NCOR2; |
| | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKC1; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1, |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| | PARK7; PSEN1; PARK2; APP; CASP3 |
| Notch Signaling | HES1; JAG1; NUMB; NOTCH4; ADAM17; NOTCH2; |
| | PSEN1; NOTCH3; NOTCH1; DLL4 |
| Endoplasmic Reticulum Stress | HSPA5; MAPK8; XBP1; TRAF2; ATF6; CASP9; ATF4; |
| Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta | GNAS; GNAQ; PPP2R1A; GNB2L1; PPP2CA; PPP1CC; |
| Adrenergic Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRKIB |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid Metabolism | PRDX6; GRN; YWHAZ; CYP1B1 |
| Circadian Rhythm Signaling | CSNK1E; CREB1; ATF4; NR1D1 |
| Coagulation System | BDKRB1; F2R; SERPINE1; F3 |
| Dopamine Receptor Signaling | PPP2R1A; PPP2CA; PPP1CC; PPP2R5C |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation Pathway | CALR; B2M |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics by | GSTP1; CYP1B1 |
| Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid Metabolism | PRMT5; AHCY |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor Signaling | GNB2L1 |
| NRF2-mediated Oxidative | PRDX1 |
| Stress Response | |
| Pentose Phosphate Pathway | GPI |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and Isoleucine | ALDH1A1 |
| Degradation | |
| Glycine, Serine and Threonine | CHKA |
| Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf, Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4fl or Brn3a); Numb; Reln |

Any one of the systems and methods disclosed herein can be utilized to treat a target cell, a target tissue, a target condition, or a target disease of a subject.

A target disease can be a viral, bacterial, and/or parasitic infection; inflammatory and/or autoimmune disease; or neoplasm such as a cancer and/or tumor.

A target cell can be a diseased cell. A diseased cell can have altered metabolic, gene expression, and/or morphologic features. A diseased cell can be a cancer cell, a diabetic cell, and an apoptotic cell. A diseased cell can be a cell from a diseased subject. Exemplary diseases can include blood disorders, cancers, metabolic disorders, eye disorders, organ disorders, musculoskeletal disorders, cardiac disease, and the like. A variety of target cells can be killed using any one of the methods or compositions disclosed herein. A target cell can include a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem (iPS) cells) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can comprise a target nucleic acid. A target cell can be in a living organism. A target cell can be a genetically modified cell. A target cell can be a host cell.

A target cell can be a totipotent stem cell, however, in some embodiments of this disclosure, the term “cell” may be used but may not refer to a totipotent stem cell. A target cell can be a plant cell, but in some embodiments of this disclosure, the term “cell” may be used but may not refer to a plant cell. A target cell can be a pluripotent cell. For example, a target cell can be a pluripotent hematopoietic cell that can differentiate into other cells in the hematopoietic cell lineage but may not be able to differentiate into any other non-hematopoietic cell. A target cell may be able to develop into a whole organism. A target cell may or may not be able to develop into a whole organism. A target cell may be a whole organism.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and a apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

Non-limiting examples of cells which can be target cells include, but are not limited to, lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intra-glomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

Of particular interest are cancer cells. In some embodiments, the target cell is a cancer cell. Non-limiting examples of cancer cells include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell pro-lymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

In some cases, the subject can have or can be suspected of having an autoimmune disease. Non-limiting examples of an autoimmune disease can include acute disseminated encephalomyelitis (ADEM), acute necrotizing hemorrhagic leukoen-cephalitis, Addison's disease, agammaglobulinemia, allergic asthma, allergic rhinitis, alopecia areata, amyloidosis, ankylosing spondylitis, antibody-mediated transplantation rejection, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopeni purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogam-maglobulinemia, hypergammaglobulinemia, idiopathic thrombocytopenia purpura (ITP), IgA nephropathy, IgG4-related sclerosing disease, immunoregulatory lipoproteins, inclusion body myositis, inflammatory bowel disease, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), lupus (SLE), lyme disease, Meniere's disease, microscopic polyangiitis, mixed connective tissue disease (MCTD), monoclonal gammopathy of undetermined significance (MGUS), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar de-generation, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis (Granulomatosis with Polyangiitis (GPA)).

In some cases, the autoimmune disease comprises one or more members selected from the group comprising rheumatoid arthritis, type 1 diabetes, systemic lupus erythematosus (lupus or SLE), myasthenia gravis, multiple sclerosis, scleroderma, Addison's Disease, bullous pemphigoid, pemphigus vulgaris, Guillain-Barré syndrome, Sjogren syndrome, dermatomyositis, thrombotic thrombocytopeniaurpura, hypergammaglobulinemia, monoclonal gammopathy of undetermined significance (MGUS), Waldenstrom's macroglobulinemia (WM), chronic inflammatory demyelinating polyradiculoneuropathy (CIDP), Hashimoto's Encephalopathy (HE), Hashimoto's Thyroiditis, Graves' Disease, Wegener's Granulomatosis, and antibody-mediated transplantation rejection (e.g., for tissue transplants such as renal transplant). In examples, the autoimmune disease can be type 1 diabetes, lupus, or rheumatoid arthritis.

In some cases, the target cells form a tumor (i.e., a solid tumor). A tumor treated with the methods herein can result in stabilized tumor growth (e.g., one or more tumors do not increase more than 1%, 5%, 10%, 15%, or 20% in size, and/or do not metastasize). In some cases, a tumor is stabilized for at least about 1, 2, 3, 4, 5, 6, 7, 8, 9, 10, 11, 12, or more weeks. In some cases, a tumor is stabilized for at least about 1, 2, 3, 4, 5, 6, 7, 8, 9, 10, 11, 12, or more months. In some cases, a tumor is stabilized for at least about 1, 2, 3, 4, 5, 6, 7, 8, 9, 10, or more years. In some cases, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95% or more. In some cases, the tumor is completely eliminated, or reduced below a level of detection. In some cases, a subject remains tumor free (e.g. in remission) for at least about 1, 2, 3, 4, 5, 6, 7, 8, 9, 10, 11, 12, or more weeks following treatment. In some cases, a subject remains tumor free for at least about 1, 2, 3, 4, 5, 6, 7, 8, 9, 10, 11, 12, or more months following treatment. In some cases, a subject remains tumor free for at least about 1, 2, 3, 4, 5, 6, 7, 8, 9, 10, or more years after treatment.

### EXAMPLES

### Example 1: Regulating Endogenous Target Proteins

Jurkat cells were transduced with lentivirus containing efla-dCas9-VPR-Q8 (hereinafter the “Q8”) and the Q8-positive cells were sorted subsequently (e.g., about 1 week later). The sorted Q8-positive cells were then sorted again (e.g., about 2 weeks later) to establish a dCas9-VPR expressing cell line. These cells (e.g., about 200,000 cells per reaction) were then transfected with sgRNA (e.g., about 250-500 ng of sgRNA) using a transfection agent. Cells were plated (e.g., in a 96 well plate) in media (e.g., RPMI1640+10% FCS) and incubated at 37° C. for a period of time (e.g., 48-72 hours) prior to gene expression analysis.

Gene expressions of the target proteins were measured with SYBR green qPCR using the delta delta Ct method, using the primers (e.g., forward (F) primer, reverse (R) primer) provided in Table 7.

TABLE 7

| Target | F primer | R primer |
|---|---|---|
| qPCR_ | GTCTCCTCTGACTTCAACAGC | ACCACCCTGTTGCTGTAGCC |
| GAPDH | G | AA |
| qPCR_ | CCTTGAAAGCTCAGAACTCG | TGCTGCGTTAGCATGAGTTG |
| JUN | GAG | GC |
| qPCRf_ | CAGCTTAGCCAGGTGGAAAT | GTCGTTGGAGATGACAAGTT |
| ID3 | CC | CCG |
| qPCRf_ | GTCCAACAATGTGACCCAGA | ACCTCAACGATATGCAGCCG |
| TBX21 | T | |
| qPCRf_ | TAGAGACAAACTGTGAGTGG | GGGCATGTTAGTCTGTGTTT |
| IL21 | TCA | CTG |
| qPCRf_ | CGCTACCTTTGGCGAAGTCT | CTGGCTCTGTATGCTGCGAG |
| TOX | CT | TT |
| qPCRf_ | CTCAGGAAGAGGAGTCGGAA | ACACAGGCTTCTGCGGCTCA |
| TOX2 | GT | TT |
| qPCRf_ | TTCGCCCTTAGCGTGAAGAT | TAGTGCTCCAGCAGCTCGAA |
| SOCS1 | GG | GA |
| qPCRf_ | TGTGACCGAGTCCTCTGGAA | GCCTCAAATGTGGCAAAGAC |
| SHIP1 | GT | AGG |
| qPCRf_ | TATTGCCGCCCAGAAGAGC | GCTTGATCTCCTTGCGTAGA |
| BATF | | G |
| qPCRf_ | CCACTGAAAAAGATGAGTAT | CCAATCCAAATGCGGCATCT |
| B2M | GCCT | TCA |

a. ID3

Positions of target polynucleotide sequences of a plurality of guide RNAs with respect to a gene encoding ID3 are shown in FIG. 5A, and sequences of the plurality of guide RNAs against ID3 are provided in FIG. 5B (top).

Enhanced expression of endogenous ID3 in the Jurkat cells, upon activation by the system, as disclosed herein, comprising the Q8 and one of the plurality of guide RNAs against ID3 is shown in FIG. 5B (bottom). In some cases, use of such system promoted enhanced expression level of endogenous ID3 by about 25-fold (e.g., ID3_UP_gR61r), about 23-fold (e.g., ID3_UP_gR31r), or about 5-fold (e.g., ID3_UP_gR62r), as compare to control Jurket cells with a control, e.g., a control gRNA which binds to a different location of the ID3 gene or which does not exhibit specific binding affinity to the ID3 gene.

b. c-Jun

Positions of target polynucleotide sequences of a plurality of guide RNAs with respect to a gene encoding c-Jun are shown in FIG. 6A, and sequences of the plurality of guide RNAs against c-Jun are provided in FIG. 6B (top).

Enhanced expression of endogenous c-Jun in the Jurkat cells, upon activation by the system, as disclosed herein, comprising the Q8 and one of the plurality of guide RNAs against c-Jun is shown in FIG. 6B (bottom). In some cases, use of such system promoted enhanced expression level of endogenous c-Jun by about 19-fold (e.g., JUN_UP_gR94f), about 13-fold (e.g., JUN_UP_gR31f or JUN_UP_gR53f), or about 4-fold (e.g., JUN_UP_gR53f), as compare to control Jurket cells with a control, e.g., a control gRNA which binds to a different location of the c-Jun gene or which does not exhibit specific binding affinity to the c-Jun gene.

c. TBX21

Positions of target polynucleotide sequences of a plurality of guide RNAs with respect to a gene encoding TBX21 are shown in FIG. 7A, and sequences of the plurality of guide RNAs against TBX21 are provided in FIG. 7B (top).

Enhanced expression of endogenous TBX21 in the Jurkat cells, upon activation by the system, as disclosed herein, comprising the Q8 and one of the plurality of guide RNAs against TBX21 is shown in FIG. 7B (bottom). In some cases, use of such system promoted enhanced expression level of endogenous TBX21 by about 400-fold (e.g., TBX21_UP_gR32r), about 250-fold (e.g., TBX21_UP_gR8r), or about 140-fold (e.g., TBX21_UP_gR77f), or about 120-fold (e.g., TBX21_UP_gR64r), relative to control Jurket cells with a control, e.g., a control gRNA which binds to a different location of the TBX21 gene or which does not exhibit specific binding affinity to the TBX21 gene.

d. IL-21

Positions of target polynucleotide sequences of a plurality of guide RNAs with respect to a gene encoding IL-21 are shown in FIG. 8A, and sequences of the plurality of guide RNAs against IL-21 are provided in FIG. 8B (top).

Enhanced expression of endogenous IL-21 in the Jurkat cells, upon activation by the system, as disclosed herein, comprising the Q8 and one of the plurality of guide RNAs against IL-21 is shown in FIG. 8B (bottom). In some cases, use of such system promoted enhanced expression level of endogenous IL-21 by about 10-fold (e.g., IL-21_UP_gR8r), about 100-fold (e.g., IL-21_UP_gR16r), or about 1,000-fold (e.g., IL-21_UP_gR42f), relative to control Jurket cells with a control gRNA (e.g., IL21_UP_gR92f) which binds to a different location of the IL-21 gene or which does not exhibit specific binding affinity to the IL-21 gene.

e. TOX1

Positions of target polynucleotide sequences of a plurality of guide RNAs with respect to a gene encoding TOX1 are shown in FIG. 9A, and sequences of the plurality of guide RNAs against TOX1 are provided in FIG. 9B (top).

Expression of endogenous TOX1 in the Jurkat cells, upon repression by the system, as disclosed herein, comprising the Q8 and one of the plurality of guide RNAs against TOX1 is shown in FIG. 9B (bottom). In some cases, use of such system promoted decreased expression level of endogenous TOX1, as indicated an expression level of the endogenous TOX1 that is about 0.4 (e.g., TOX_1), about 0.5 (e.g., TOX_2), or between about 0.7 and about 0.8 (e.g., TOX_3 and TOX_4), relative to that in control Jurket cells with a control gRNA which binds to a different location of the TOX1 gene or which does not exhibit specific binding affinity to the TOX1 gene.

f. TOX2

Positions of target polynucleotide sequences of a plurality of guide RNAs with respect to a gene encoding TOX2 are shown in FIG. 10A, and sequences of the plurality of guide RNAs against TOX2 are provided in FIG. 10B (top).

Expression of endogenous TOX2 in the Jurkat cells, upon repression by the system, as disclosed herein, comprising the Q8 and one of the plurality of guide RNAs against TOX2 is shown in FIG. 10B (bottom). In some cases, use of such system promoted decreased expression level of endogenous TOX2, as indicated an expression level of the endogenous TOX2 that is about 0.1 (e.g., TOX2_3), about 0.2 (e.g., TOX2_1), about 0.5 (e.g., TOX2_4), or about 0.8 (e.g., TOX2_2), relative to that in control Jurket cells with a control gRNA which binds to a different location of the TOX2 gene or which does not exhibit specific binding affinity to the TOX2 gene.

g. SHIP1

Positions of target polynucleotide sequences of a plurality of guide RNAs with respect to a gene encoding SHIP1 are shown in FIG. 11A, and sequences of the plurality of guide RNAs against SHIP1 are provided in FIG. 11B (top).

Expression of endogenous SHIP1 in the Jurkat cells, upon repression by the system, as disclosed herein, comprising the Q8 and one of the plurality of guide RNAs against SHIP1 is shown in FIG. 11B (bottom). In some cases, use of such system promoted decreased expression level of endogenous SHIP1, as indicated an expression level of the endogenous SHIP1 that is about 0.2 (e.g., SHIPgRr7, SHIPgR32f, or SHIPgR49r) or about 0.3 (e.g., SHIPgR60f), relative to that in control Jurket cells with a control gRNA which binds to a different location of the SHIP1 gene or or which does not exhibit specific binding affinity to the SHIP1 gene.

h. B2M

Positions of target polynucleotide sequences of a plurality of guide RNAs with respect to a gene encoding B2M are shown in FIG. 12A, and sequences of the plurality of guide RNAs against B2M are provided in FIG. 12B (top).

Expression of endogenous B2M in the Jurkat cells, upon repression by the system, as disclosed herein, comprising the Q8 and one of the plurality of guide RNAs against B2M is shown in FIG. 12B (bottom). In some cases, use of such system promoted decreased expression level of endogenous B2M, as indicated an expression level of the endogenous B2M that is about 0.7 (e.g., B2M_d_gR56f), about 0.6 (e.g., B2M_d_gR21r), or about 0.4 (e.g., B2M_gR21r), relative to that in control Jurket cells with a control gRNA which binds to a different location of the B2M gene (e.g., B2M_d_gR248f) or or which does not exhibit specific binding affinity to the B2M gene.

i. BATF

Positions of target polynucleotide sequences of a plurality of guide RNAs with respect to a gene encoding BATF are shown in FIG. 13A, and sequences of the plurality of guide RNAs against BATF are provided in FIG. 13B (top).

Expression of endogenous BATF in the Jurkat cells, upon repression by the system, as disclosed herein, comprising the Q8 and one of the plurality of guide RNAs against BATF is shown in FIG. 13B (bottom). In some cases, use of such system promoted decreased expression level of endogenous BATF, as indicated an expression level of the endogenous BATF that is about 0.6 (e.g., BATF_d_gR41f or BATF_d_gR56f), about 0.5 (e.g., BATF_d_gR62f), or about 0.3 (e.g., BATF_d_gR22f), relative to that in control Jurket cells with a control gRNA which binds to a different location of the BATF gene or which does not exhibit specific binding affinity to the BATF gene.

j. SOCS1

Positions of target polynucleotide sequences of a plurality of guide RNAs with respect to a gene encoding SOCS1 are shown in FIG. 14A, and sequences of the plurality of guide RNAs against SOCS1 are provided in FIG. 14B (top).

Expression of endogenous SOCS1 in the Jurkat cells, upon repression by the system, as disclosed herein, comprising the Q8 and one of the plurality of guide RNAs against SOCS1 is shown in FIG. 14B (bottom). In some cases, use of such system promoted decreased expression level of endogenous SOCS1, as indicated an expression level of the endogenous SOCS1 that is about 0.9 (e.g., SOCS1_d_gR20f), about 0.6 (e.g., SOCS1_d_gR52r), about 0.4 (e.g., SOCS1_d_gR43f), or about 0.3 (e.g., SOCS1_d_gR53f), relative to that in control Jurket cells with a control gRNA which binds to a different location of the SOCS1 gene or which does not exhibit specific binding affinity to the SOCS1 gene.

k. TGFbR2

TGF beta (TGFb) can be a pleiotropic cytokine, which can be secreted by tumor cells in the tumor micro-environment (TME). T cells can express a receptor for TGFb, e.g., TGFbR2. Upon binding of TGFb to TGFbR (e.g., TGFbR2), downstream signaling of TGFbR can prevent T cells from differentiating into T cell sub-types (e.g., Th1 cells) and reduce anti-tumor responses (e.g., IFNg secretion) and/or tumor cytotoxicity.

To screen for guide nucleic acid molecules usable for repressing signaling or activity of TGFbR2, Jurkat cells were engineered to express dCAS9-KRAB and were transfected with plasmids encoding different TGFbR2-targeting gRNA. After transfection (e.g., 48-72 hours after transfection), cells were collected and stained with anti-TGFbR2-PE antibody and expression was determined by flow cytometry. This screen was repeated (e.g., 3 times) and the most effective gRNAs were selected. The top lead gRNAs were located mainly in the area 50-100 base pairs (bp) downstream of the TSS, or 30-70 bp upstream of the TSS. Positions of target polynucleotide sequences of a plurality of guide RNAs with respect to a gene encoding TGFbR2 are shown in FIG. 15A.

Expression of endogenous TGFbR2 in the Jurkat cells, upon repression by the system, as disclosed herein, comprising the dCAS9-KRAB and one of the plurality of guide RNAs against TGFbR2 is shown in FIG. 15B. In some cases, use of such system promoted decreased expression level of endogenous TGFbR2, as indicated an expression level of the endogenous TGFbR2 that is between about 35% and about 20% lower than that in control Jurket cells with a control gRNA which binds to a different location of the TGFbR2 gene or which does not exhibit specific binding affinity to the TGFbR2 gene.

### Embodiments

The following non-limiting embodiments provide illustrative examples of the invention, but do not limit the scope of the invention.

Embodiment 1. A system for regulating expression or activity of a target protein of a cell, the system comprising:
an actuator moiety capable of complexing with a target polynucleotide sequence in the cell, wherein the actuator moiety is heterologous to the cell, and wherein the target polynucleotide sequence is endogenous to the cell,
wherein the complexing effects a change in the expression or activity of the target protein by at least about 10% as compared to that in a control cell, wherein the complexing is sufficient to effect the change without editing the target polynucleotide sequence, and
wherein the target protein comprises one or more members selected from the group consisting of thymocyte selection-associated high mobility group box protein (TOX), suppressor of cytokine signaling (SOCS), basic leucine zipper transcription factor ATF-like (BATF), inhibitor of DNA binding/differentiation (ID), T-box transcription factor (TBX), c-Jun,
optionally wherein:
(1) the target protein is the TOX, optionally wherein the TOX comprises TOX1 or TOX2; and/or
(2) the target protein is the SOCS, optionally wherein the SOCS comprises SOCS1; and/or
(3) the target protein is the BATF; and/or
(4) the target protein is the ID, optionally wherein the ID comprises ID3; and/or
(5) the target protein is the TBX, optionally wherein the TBX is TBX21 (T-Bet); and/or
(6) the target protein is the c-Jun; and/or
(7) the actuator moiety is activatable for the complexing upon exposure of the cell to an external stimulus.

Embodiment 2. A system for regulating expression or activity of a target protein of a cell, the system comprising:
an actuator moiety capable of complexing with a target polynucleotide sequence in the cell, wherein the actuator moiety is heterologous to the cell and is activatable for the complexing upon exposure of the cell to an external stimulus, and wherein the target polynucleotide sequence is endogenous to the cell,
wherein, upon the exposure, the actuator moiety is activated for the complexing to effect a change in the expression or activity of the target protein, wherein the complexing is sufficient to effect the change without editing the target polynucleotide sequence, and
wherein the target protein comprises Src homology 2 domain containing inositol phosphatase (SHIP) or beta-2-microglobulin (B2M), and TGF beta receptor (TGFbR),
optionally wherein:
(1) upon the exposure, the actuator moiety is activated for the complexing to effect the change in the expression or activity of the target protein by at least about 10% as compared to that in a control cell; and/or
(2) the target protein is the SHIP, optionally wherein the SHIP is SHIP1; and/or
(3) the target protein is the B2M; and/or
(4) the target protein is the TGFbR, optionally wherein the TGFBR is TGFbR2.

Embodiment 3. The system of Embodiment 1 or Embodiment 2, further optionally wherein:
(1) the target protein is not a secretory protein; and/or
(2) the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein; and/or
(3) the target polynucleotide sequence is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base upstream of the TSS of the gene encoding the target protein; and/or
(4) the target polynucleotide sequence is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base downstream of the TSS of the gene encoding the target protein; and/or
(5) the change is a decrease in the expression or activity of the target protein by at least about 10%, 20%, 30%, 40%, 50%, 60%, 70%, 80%, 90%, 95%, 99%, or more as compared to that in the control cell; and/or
(6) the change is an increase in the expression or activity of the target protein by at least about 10%, 20%, 30%, 40%, 50%, 60%, 70%, 80%, 90%, 100%, 200%, 300%, 400%, 500%, or more as compared to that in the control cell; and/or
(7) the cell is an immune cell, and the change in the expression or activity of the target protein promotes maintenance of stemness of the immune cell, survival of the immune cell, and/or expansion of the immune cell; and/or
(8) the cell is an immune cell, and the change in the expression or activity of the target protein promotes enhanced cytokine production by the engineered immune cell, enhanced cytotoxicity of the engineered immune cell against a population of target cells, and/or reduced exhaustion of the immune cell; and/or
(9) the external stimulus is a ligand, and the system comprises a chimeric receptor polypeptide (receptor) that undergoes a modification upon binding to the ligand, wherein the actuator moiety is activatable upon the receptor modification; and/or
(10) the activation of the actuator moiety comprises (1) release of the actuator moiety from a substrate or (2) a modification of the actuator moiety; and/or
(11) the actuator moiety comprises a nucleic acid-guided actuator moiety, and wherein the system further comprises a guide nucleic acid that complexes with the actuator moiety; and/or
(12) the guide nucleic acid comprises a guide ribonucleic acid (RNA); and/or
(13) the system comprises two or more guide nucleic acids having complementarity to different target polynucleotide sequences; and/or
(14) the actuator moiety comprises an effector domain that is configured to regulate the expression or activity of the target protein; and/or
(15) the effector domain is selected from the group consisting of a cleavage domain,
an epigenetic modification domain, a transcriptional activation domain, or a transcriptional repressor domain, further optionally wherein:
(a) the effector domain is a transcriptional activation domain; and/or
(b) the effector domain is a transcriptional repressor domain; and/or
(16) the actuator moiety comprises a heterologous endonuclease or a variant thereof; and/or
(17) the modification is a conformational change or chemical modification; and/or
(18) the cell is an immune cell; and/or
(19) the cell is a T cell or NK cell.

Embodiment 4. A population of engineered cells, wherein each engineered cell of the population comprises the system of Embodiments 1-3

Embodiment 5. A composition comprising the population of engineered cells of Embodiment 4, optionally wherein the composition further comprises a co-therapeutic agent.

Embodiment 6. A system comprising a guide nucleic acid molecule designed to bind a target polynucleotide sequence for regulating expression or activity of a target protein of a cell, wherein the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein,
wherein the target protein is selected from the group consisting of thymocyte selection-associated high mobility group box protein (TOX), suppressor of cytokine signaling (SOCS), Src homology 2 domain containing inositol phosphatase (SHIP), basic leucine zipper transcription factor ATF-like (BATF), beta-2-microglobulin (B2M), inhibitor of DNA binding/differentiation (ID), T-box transcription factor (TBX), c-Jun, and TGF beta receptor (TGFbR), optionally wherein:
(1) the guide nucleic acid molecule is capable of recruiting an actuator moiety to the target polynucleotide sequence, to regulate expression or activity of the target protein, and wherein the system further comprises the actuator moiety; and/or
(2) the actuator moiety comprises a heterologous endonuclease or a variant thereof; and/or
(3) the gene encoding the target protein is endogenous to the cell; and/or
(4) the TSS is endogenous to the cell.

Embodiment 7. A system comprising an actuator moiety capable of binding a target polynucleotide sequence for regulating expression or activity of a target protein of a cell, wherein the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein,
wherein the target protein is selected from the group consisting of thymocyte selection-associated high mobility group box protein (TOX), suppressor of cytokine signaling (SOCS), Src homology 2 domain containing inositol phosphatase (SHIP), basic leucine zipper transcription factor ATF-like (BATF), beta-2-microglobulin (B2M), inhibitor of DNA binding/differentiation (ID), T-box transcription factor (TBX), c-Jun, and TGF beta receptor (TGFbR), optionally wherein:
(1) the actuator moiety comprises a heterologous endonuclease or a variant thereof; and/or
(2) the gene encoding the target protein is endogenous to the cell; and/or
(3) the TSS is endogenous to the cell.

Embodiment 8. A method for regulating expression or activity of a target protein of a cell, comprising:
(a) forming a complex comprising an actuator moiety and a target polynucleotide sequence in the cell, wherein the actuator moiety is heterologous to the cell, and wherein the target polynucleotide sequence is endogenous to the cell;
(b) in response to the forming, inducing a change in the expression or activity of the target protein by at least about 10% as compared to that in a control cell, wherein the forming of the complex is sufficient to effect the change without editing the target polynucleotide sequence,
wherein the target protein comprises one or more members selected from the group consisting of thymocyte selection-associated high mobility group box protein (TOX), suppressor of cytokine signaling (SOCS), basic leucine zipper transcription factor ATF-like (BATF), inhibitor of DNA binding/differentiation (ID), T-box transcription factor (TBX), c-Jun, and TGF beta receptor (TGFbR),
optionally wherein:
(1) the target protein is the TOX, optionally wherein the TOX comprises TOX1 or TOX2; and/or
(2) the target protein is the SOCS, optionally wherein the SOCS comprises SOCS1; and/or
(3) the target protein is the BATF; and/or
(4) the target protein is the ID, optionally wherein the ID comprises ID3; and/or
(5) the target protein is the TBX, optionally wherein the TBX is TBX21 (T-Bet); and/or
(6) the target protein is the c-Jun; and/or
(7) the target protein is the TGFbR, optionally wherein the TGFbR is TGFbR2; and/or
(8) the target protein is not a cytokine, optionally wherein the target protein is not a secretory protein; and/or
(9) the actuator moiety is activatable for the complexing upon exposure of the cell to an external stimulus.

Embodiment 9. A method for regulating expression or activity of a target protein of a cell, comprising:
(a) exposing the cell to an external stimulus to activate an actuator moiety to complex with a target polynucleotide sequence in the cell, wherein the actuator moiety is heterologous to the cell, and wherein the target polynucleotide sequence is endogenous to the cell; and
(b) in response to the complexing, inducing a change in the expression or activity of the target protein, wherein the forming of the complex is sufficient to effect the change without editing the target polynucleotide sequence,
wherein the target protein comprises Src homology 2 domain containing inositol phosphatase (SHIP) or beta-2-microglobulin (B2M), optionally wherein:
(1) in response to the complexing, the induced change in the expression or activity of the target protein is at least about 10% as compared to that in a control cell; and/or
(2) the target protein is the SHIP, optionally wherein the SHIP is SHIP1; and/or
(3) the target protein is the B2M; and/or
(4) the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein; and/or
(5) the target polynucleotide sequence is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base upstream of the TSS of the gene encoding the target protein; and/or
(6) the target polynucleotide sequence is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base downstream of the TSS of the gene encoding the target protein; and/or
(7) the change is a decrease in the expression or activity of the target protein by at least about 10%, 20%, 30%, 40%, 50%, 60%, 70%, 80%, 90%, 95%, 99%, or more as compared to that in the control cell; and/or
(8) the change is an increase in the expression or activity of the target protein by at least about 10%, 20%, 30%, 40%, 50%, 60%, 70%, 80%, 90%, 100%, 200%, 300%, 400%, 500%, or more as compared to that in the control cell; and/or
(9) the cell is an immune cell, and the change in the expression or activity of the target protein promotes maintenance of stemness of the immune cell, survival of the immune cell, and/or expansion of the immune cell; and/or
(10) the cell is an immune cell, and the change in the expression or activity of the target protein promotes enhanced cytokine production by the engineered immune cell, enhanced cytotoxicity of the engineered immune cell against a population of target cells, and/or reduced exhaustion of the immune cell; and/or
(11) the external stimulus is a ligand, and the cell comprises a chimeric receptor polypeptide (receptor) that undergoes a modification upon binding to the ligand, wherein the actuator moiety is activatable upon the receptor modification; and/or
(12) the activation of the actuator moiety comprises (1) release of the actuator moiety from a substrate or (2) a modification of the actuator moiety; and/or
(13) the actuator moiety comprises a nucleic acid-guided actuator moiety, and wherein the method further comprises contacting the cell with a guide nucleic acid that complexes with the actuator moiety; and/or
(14) the guide nucleic acid comprises a guide ribonucleic acid (RNA); and/or
(15) the guide nucleic acid comprises two or more guide nucleic acids having complementarity to different target polynucleotide sequences; and/or
(16) the actuator moiety comprises an effector domain that is configured to regulate the expression or activity of the target protein; and/or
(17) the effector domain is selected from the group consisting of a cleavage domain, an epigenetic modification domain, a transcriptional activation domain, or a transcriptional repressor domain, further optionally wherein:
(a) the effector domain is a transcriptional activation domain; and/or
(b) the effector domain is a transcriptional repressor domain; and/or
(18) the actuator moiety comprises a heterologous endonuclease or a variant thereof; and/or
(19) the modification is a conformational change or chemical modification; and/or
(20) the cell is an immune cell; and/or
(21) the cell is a T cell or NK cell; and/or
(22) further comprising administering the cell to a subject in need thereof; and/or
(23) the cell is autologous or allogeneic to the subject; and/or
(24) further comprising administering a co-therapeutic agent to the subject; and/or
(25) the subject is a mammal; and/or
(26) the subject is a human.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and il-lustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A system for regulating expression or activity of a target protein of a cell, the system comprising:
an actuator moiety capable of complexing with a target polynucleotide sequence in the cell, wherein the actuator moiety is heterologous to the cell, and wherein the target polynucleotide sequence is endogenous to the cell,
wherein the complexing effects a change in the expression or activity of the target protein by at least about 10% as compared to that in a control cell, wherein the complexing is sufficient to effect the change without editing the target polynucleotide sequence, and
wherein the target protein comprises one or more members selected from the group consisting of thymocyte selection-associated high mobility group box protein (TOX), suppressor of cytokine signaling (SOCS), basic leucine zipper transcription factor ATF-like (BATF), inhibitor of DNA binding/differentiation (ID), T-box transcription factor (TBX), c-Jun.

2. The system of claim 1, wherein the target protein is the TOX.

3. The system of claim 2, wherein the TOX comprises TOX1 or TOX2.

4. The system of claim 1, wherein the target protein is the SOCS.

5. The system of claim 3, wherein the SOCS comprises SOCS1.

6. The system of claim 1, wherein the target protein is the BATF.

7. The system of claim 1, wherein the target protein is the ID.

8. The system of claim 7, wherein the ID comprises ID3.

9. The system of claim 1, wherein the target protein is the TBX.

10. The system of claim 9, wherein the TBX is TBX21 (T-Bet).

11. The system of claim 1, wherein the target protein is the c-Jun.

12. The system of any one of the preceding claims, wherein the actuator moiety is activatable for the complexing upon exposure of the cell to an external stimulus.

13. A system for regulating expression or activity of a target protein of a cell, the system comprising:
an actuator moiety capable of complexing with a target polynucleotide sequence in the cell, wherein the actuator moiety is heterologous to the cell and is activatable for the complexing upon exposure of the cell to an external stimulus, and wherein the target polynucleotide sequence is endogenous to the cell,
wherein, upon the exposure, the actuator moiety is activated for the complexing to effect a change in the expression or activity of the target protein, wherein the complexing is sufficient to effect the change without editing the target polynucleotide sequence, and
wherein the target protein comprises Src homology 2 domain containing inositol phosphatase (SHIP) or beta-2-microglobulin (B2M), and TGF beta receptor (TGFbR).

14. The system of claim 13, wherein, upon the exposure, the actuator moiety is activated for the complexing to effect the change in the expression or activity of the target protein by at least about 10% as compared to that in a control cell.

15. The system of claim 13, wherein the target protein is the SHIP.

16. The system of claim 15, wherein the SHIP is SHIP1.

17. The system of claim 13, wherein the target protein is the B2M.

18. The system of claim 13, wherein the target protein is the TGFbR.

19. The system of claim 18, wherein the TGFBR is TGFbR2.

20. The system of any one of the preceding claims, wherein the target protein is not a cytokine.

21. The system of any one of the preceding claims, wherein the target protein is not a secretory protein.

22. The system of any one of the preceding claims, wherein the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein.

23. The system of any one of the preceding claims, wherein the target polynucleotide sequence is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base upstream of the TSS of the gene encoding the target protein.

24. The system of any one of the preceding claims, wherein the target polynucleotide sequence is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base downstream of the TSS of the gene encoding the target protein.

25. The system of any one of the preceding claims, wherein the change is a decrease in the expression or activity of the target protein by at least about 10%, 20%, 30%, 40%, 50%, 60%, 70%, 80%, 90%, 95%, 99%, or more as compared to that in the control cell.

26. The system of any one of the preceding claims, wherein the change is an increase in the expression or activity of the target protein by at least about 10%, 20%, 30%, 40%, 50%, 60%, 70%, 80%, 90%, 100%, 200%, 300%, 400%, 500%, or more as compared to that in the control cell.

27. The system of any one of the preceding claims, wherein the cell is an immune cell, and the change in the expression or activity of the target protein promotes maintenance of stemness of the immune cell, survival of the immune cell, and/or expansion of the immune cell.

28. The system of any one of the preceding claims, wherein, the cell is an immune cell, and the change in the expression or activity of the target protein promotes enhanced cytokine production by the engineered immune cell, enhanced cytotoxicity of the engineered immune cell against a population of target cells, and/or reduced exhaustion of the immune cell.

29. The system of any one of the preceding claims, wherein the external stimulus is a ligand, and the system comprises:
a chimeric receptor polypeptide (receptor) that undergoes a modification upon binding to the ligand, wherein the actuator moiety is activatable upon the receptor modification.

30. The system of any one of the preceding claims, wherein the activation of the actuator moiety comprises (1) release of the actuator moiety from a substrate or (2) a modification of the actuator moiety.

31. The system of any one of the preceding claims, wherein the actuator moiety comprises a nucleic acid-guided actuator moiety, and wherein the system further comprises a guide nucleic acid that complexes with the actuator moiety.

32. The system of any one of the preceding claims, wherein the guide nucleic acid comprises a guide ribonucleic acid (RNA).

33. The system of any one of the preceding claims, comprising two or more guide nucleic acids having complementarity to different target polynucleotide sequences.

34. The system of any one of the preceding claims, wherein the actuator moiety comprises an effector domain that is configured to regulate the expression or activity of the target protein.

35. The system of claim 34, wherein the effector domain is selected from the group consisting of a cleavage domain, an epigenetic modification domain, a transcriptional activation domain, or a transcriptional repressor domain.

36. The system of claim 35, wherein the effector domain is a transcriptional activation domain.

37. The system of claim 35, wherein the effector domain is a transcriptional repressor domain.

38. The system of any one of the preceding claims, wherein the actuator moiety comprises a heterologous endonuclease or a variant thereof.

39. The system of any one of the preceding claims, wherein the modification is a conformational change or chemical modification.

40. The system of any one of the preceding claims, wherein the cell is an immune cell.

41. The system of any one of the preceding claims, wherein the cell is a T cell or NK cell.

42. A population of engineered cells, wherein each engineered cell of the population comprises the system of any one of the preceding claims.

43. A composition comprising the population of engineered cells of claim 42.

44. The composition of claim 43, further comprising a co-therapeutic agent.

45. A system comprising a guide nucleic acid molecule designed to bind a target polynucleotide sequence for regulating expression or activity of a target protein of a cell, wherein the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein,
wherein the target protein is selected from the group consisting of thymocyte selection-associated high mobility group box protein (TOX), suppressor of cytokine signaling (SOCS), Src homology 2 domain containing inositol phosphatase (SHIP), basic leucine zipper transcription factor ATF-like (BATF), beta-2-microglobulin (B2M), inhibitor of DNA binding/differentiation (ID), T-box transcription factor (TBX), c-Jun, and TGF beta receptor (TGFbR).

46. The system of claim 45, wherein the guide nucleic acid molecule is capable of recruiting an actuator moiety to the target polynucleotide sequence, to regulate expression or activity of the target protein, and wherein the system further comprises the actuator moiety.

47. A system comprising an actuator moiety capable of binding a target polynucleotide sequence for regulating expression or activity of a target protein of a cell, wherein the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein,
wherein the target protein is selected from the group consisting of thymocyte selection-associated high mobility group box protein (TOX), suppressor of cytokine signaling (SOCS), Src homology 2 domain containing inositol phosphatase (SHIP), basic leucine zipper transcription factor ATF-like (BATF), beta-2-microglobulin (B2M), inhibitor of DNA binding/differentiation (ID), T-box transcription factor (TBX), c-Jun, and TGF beta receptor (TGFbR).

48. The system of any one of the preceding claims, wherein the actuator moiety comprises a heterologous endonuclease or a variant thereof.

49. The system of any one of the preceding claims, wherein the gene encoding the target protein is endogenous to the cell.

50. The system of any one of the preceding claims, wherein the TSS is endogenous to the cell.

51. A method for regulating expression or activity of a target protein of a cell, comprising:
(a) forming a complex comprising an actuator moiety and a target polynucleotide sequence in the cell, wherein the actuator moiety is heterologous to the cell, and wherein the target polynucleotide sequence is endogenous to the cell;
(b) in response to the forming, inducing a change in the expression or activity of the target protein by at least about 10% as compared to that in a control cell, wherein the forming of the complex is sufficient to effect the change without editing the target polynucleotide sequence, wherein the target protein comprises one or more members selected from the group consisting of thymocyte selection-associated high mobility group box protein (TOX), suppressor of cytokine signaling (SOCS), basic leucine zipper transcription factor ATF-like (BATF), inhibitor of DNA binding/differentiation (ID), T-box transcription factor (TBX), c-Jun, and TGF beta receptor (TGFbR).

52. The method of claim 51, wherein the target protein is the TOX.

53. The method of claim 52, wherein the TOX comprises TOX1 or TOX2.

54. The method of claim 51, wherein the target protein is the SOCS.

55. The method of claim 54, wherein the SOCS comprises SOCS1.

56. The method of claim 51, wherein the target protein is the BATF.

57. The method of claim 51, wherein the target protein is the ID.

58. The method of claim 57, wherein the ID comprises ID3.

59. The method of claim 51, wherein the target protein is the TBX.

60. The method of claim 59, wherein the TBX is TBX21 (T-Bet).

61. The method of claim 51, wherein the target protein is the c-Jun.

62. The method of claim 51, wherein the target protein is the TGFbR.

63. The method of claim 62, wherein the TGFbR is TGFbR2.

64. The method of claim 51, wherein the target protein is not a cytokine.

65. The method of claim 64, wherein the target protein is not a secretory protein.

66. The method of any one of the preceding claims, wherein the actuator moiety is activatable for the complexing upon exposure of the cell to an external stimulus.

67. A method for regulating expression or activity of a target protein of a cell, comprising:
(a) exposing the cell to an external stimulus to activate an actuator moiety to complex with a target polynucleotide sequence in the cell, wherein the actuator moiety is heterologous to the cell, and wherein the target polynucleotide sequence is endogenous to the cell; and
(b) in response to the complexing, inducing a change in the expression or activity of the target protein, wherein the forming of the complex is sufficient to effect the change without editing the target polynucleotide sequence,
wherein the target protein comprises Src homology 2 domain containing inositol phosphatase (SHIP) or beta-2-microglobulin (B2M).

68. The method of claim 67, wherein, in response to the complexing, the induced change in the expression or activity of the target protein is at least about 10% as compared to that in a control cell.

69. The method of claim 67, wherein the target protein is the SHIP.

70. The method of claim 69, wherein the SHIP is SHIP1.

71. The method of claim 67, wherein the target protein is the B2M.

72. The method of any one of the preceding claims, wherein the target polynucleotide sequence (i) comprises at least a portion of a transcription start site (TSS) of a gene encoding the target protein or (ii) is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base away from the TSS of the gene encoding the target protein.

73. The method of any one of the preceding claims, wherein the target polynucleotide sequence is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base upstream of the TSS of the gene encoding the target protein.

74. The method of any one of the preceding claims, wherein the target polynucleotide sequence is between about 10,000 and about 5,000 bases, between about 5,000 bases and about 4000 bases, between about 4,000 bases and about 3,000 bases, between about 3,000 bases and about 2,000 bases, between about 2,500 bases and about 2,000 bases, between about 2,000 bases and about 1,500 bases, between about 1,500 bases and about 1,000 bases, between about 1,000 bases and about 500 bases, or between about 500 bases and 1 base downstream of the TSS of the gene encoding the target protein.

75. The method of any one of the preceding claims, wherein the change is a decrease in the expression or activity of the target protein by at least about 10%, 20%, 30%, 40%, 50%, 60%, 70%, 80%, 90%, 95%, 99%, or more as compared to that in the control cell.

76. The method of any one of the preceding claims, wherein the change is an increase in the expression or activity of the target protein by at least about 10%, 20%, 30%, 40%, 50%, 60%, 70%, 80%, 90%, 100%, 200%, 300%, 400%, 500%, or more as compared to that in the control cell.

77. The method of any one of the preceding claims, wherein the cell is an immune cell, and the change in the expression or activity of the target protein promotes maintenance of stemness of the immune cell, survival of the immune cell, and/or expansion of the immune cell.

78. The method of any one of the preceding claims, wherein the cell is an immune cell, and the change in the expression or activity of the target protein promotes enhanced cytokine production by the engineered immune cell, enhanced cytotoxicity of the engineered immune cell against a population of target cells, and/or reduced exhaustion of the immune cell.

79. The method of any one of the preceding claims, wherein the external stimulus is a ligand, and the cell comprises:
a chimeric receptor polypeptide (receptor) that undergoes a modification upon binding to the ligand, wherein the actuator moiety is activatable upon the receptor modification.

80. The method of any one of the preceding claims, wherein the activation of the actuator moiety comprises (1) release of the actuator moiety from a substrate or (2) a modification of the actuator moiety.

81. The method of any one of the preceding claims, wherein the actuator moiety comprises a nucleic acid-guided actuator moiety, and wherein the method further comprises contacting the cell with a guide nucleic acid that complexes with the actuator moiety.

82. The method of any one of the preceding claims, wherein the guide nucleic acid comprises a guide ribonucleic acid (RNA).

83. The method of any one of the preceding claims, wherein the guide nucleic acid comprises two or more guide nucleic acids having complementarity to different target polynucleotide sequences.

84. The method of any one of the preceding claims, wherein the actuator moiety comprises an effector domain that is configured to regulate the expression or activity of the target protein.

85. The method of claim 84, wherein the effector domain is selected from the group consisting of a cleavage domain, an epigenetic modification domain, a transcriptional activation domain, or a transcriptional repressor domain.

86. The method of claim 85, wherein the effector domain is a transcriptional activation domain.

87. The method of claim 85, wherein the effector domain is a transcriptional repressor domain.

88. The method of any one of the preceding claims, wherein the actuator moiety comprises a heterologous endonuclease or a variant thereof.

89. The method of any one of the preceding claims, wherein the modification is a conformational change or chemical modification.

90. The method of any one of the preceding claims, wherein the cell is an immune cell.

91. The method of any one of the preceding claims, wherein the cell is a T cell or NK cell.

92. The method of any one of the preceding claims, further comprising administering the cell to a subject in need thereof.

93. The method of any one of the preceding claims, wherein the cell is autologous or allogeneic to the subject.

94. The method of any one of the preceding claims, further comprising administering a co-therapeutic agent to the subject.

95. The method of any one of the preceding claims, wherein the subject is a mammal.

96. The method of any one of the preceding claims, wherein the subject is a human.
